(19) United States
(12) Patent Application Publication
(10) Pub. No.: US 2005/0233309 A1
(43) Pub. Date: Oct. 20, 2005

(54) Kinetic process for the detection, diagnosis, processing, and monitoring of clonal cell populations

(76) Inventor: William D. Hankins, Monrovia, MD (US)

Correspondence Address: HOWARD J. GREENWALD P.C., 349 W. COMMERCIAL STREET SUITE 2490, EAST ROCHESTER, NY, 14445-2408, US

(21) Appl. No.: 11/108,310

(22) Filed: Apr. 18, 2005

Related U.S. Application Data
(60) Provisional application No. 60/563,326, filed on Apr. 19, 2004.

(51) Int. Cl. C12Q 1/00

(52) U.S. Cl. 435/4

(57) ABSTRACT

A process for identifying the developmental history of a cell. In this process, a tissue sample is obtained from a living biological organism and, thereafter, is disaggregated to produce fragments whose maximum dimension is less than about 5 millimeters; the tissue sample is preferably disaggregated within about 10 minutes of the time the tissue sample is obtained from the biological organism. Thereafter, the disaggregated tissue fragments are disposed in a sterile environment within a container; the sterile environment contains oxygen and a cell type specific viability factor.

### CROSS-REFERENCE TO RELATED PATENT APPLICATIONS

Priority for this patent application is based upon provisional patent application 60/563,326 (filed on Apr. 19, 2004). The disclosure of this United States patent application is hereby incorporated by reference into this specification.

### FIELD OF THE INVENTION

A mapping process for identifying the developmental addresses of cellular clones and their developmental histories, current status, and futures by characterizing and monitoring their response to external agents, their gene expression patterns, their morphology, and their performance.

### BACKGROUND OF THE INVENTION

Cancer is one of the main maladies affecting mankind. People have been seeking cures for cancer for many years, to little avail. With a few notable exceptions, current therapies offered by the pharmaceuticals are ineffective for the majority of patients. Therefore, if surgery does not eradicate the cancer, the patients are likely to succumb to the disease. In addition, some of the promising drugs being developed are initially effective for a particular patient, but many patients often develop drug resistance, thereby requiring different drug regimens which also eventually become ineffective.

Drug resistance often develops when some but not all of cancer cells are initially killed by the drug regimen. If a drug, or a combination of drugs, could be found that would eradicate all of the cancers cells, drug resistance would be less likely to occur.

It is an object of this invention to provide a process for identifying and characterizing the phenotypes of cancer cells so that one can more readily identify those drugs and/or drug combinations that will be most likely to eradicate such cancer cells.

It is another object of this invention to provide a molecular, cellular, and drug target mapping system for both normal and abnormal development pathways.

### SUMMARY OF THE INVENTION

In accordance with this invention, there is provided a process for identifying the developmental history of a cell, comprising the steps of (a) obtaining a tissue sample from a human being, (b) disaggregating said tissue sample to produce disaggregated fragments of tissue sample whose maximum dimension is less than about 5 millimeters, and, wherein said tissue sample is disaggregated within about 10 minutes of the time said tissue sample is obtained from said human being, and (c) disposing said disaggregated tissue fragments in a sterile environment within a container, wherein said sterile environment is comprised of oxygen and a solution comprised of at least one cell type specific viability factor.

### BRIEF DESCRIPTION OF THE DRAWINGS

The invention will described by reference to the following drawings, in which like numerals refer to like elements, and in which:

FIG. 1 is a flowchart illustrating one preferred process of the invention;

FIG. 2 is a flowchart of another preferred process of the invention;

FIG. 3 is a map of a coordinate system in which the lineage of a particular cell is traced; Hankins to improve Sun am

FIG. 4 is a schematic representation of a single ray on the coordinate system of FIG. 2;

FIG. 5 is a flow diagram of a preferred process for tissue preservation, expansion and physiological analyses in which harvested tissue is utilized;

FIG. 6 is a schematic of one preferred apparatus of the invention;

FIG. 7 is a schematic illustration of device utilized in the measurement of the optical properties of cells;

FIG. 8 is a representation of graphs illustrative of the information derived from the optical properties of cells;

FIG. 9 is a representation of graphs illustrative of the information derived from the optical properties of cells;

FIG. 10 is a representation of graphs illustrative of the information derived from the optical properties of cells;

FIG. 11 is a representation of graphs illustrative of the information derived from the optical properties of cells;

FIG. 12 is a representation of graphs illustrative of the information derived from the optical properties of cells;

FIG. 13 is a representation of graphs illustrative of the information derived from the optical properties of cells;

FIG. 14 is a flow diagram of another preferred process of the invention;

FIG. 15 is a schematic of a preferred device for measuring the transmittance of a cell culture;

FIG. 16 is a schematic of another device for measuring the optical properties of a cell culture;

FIG. 17 is a schematic of yet another device for measuring the optical properties of a cell culture;

FIG. 18 is an enlarged view of a portion of the device of FIG. 17;

FIG. 19 is an illustrative graph of one preferred process;

FIG. 20 is a schematic of a preferred embodiment of the process; and

FIG. 21 is a schematic of a preferred embodiment of the process.

### DESCRIPTION OF THE PREFERRED EMBODIMENTS

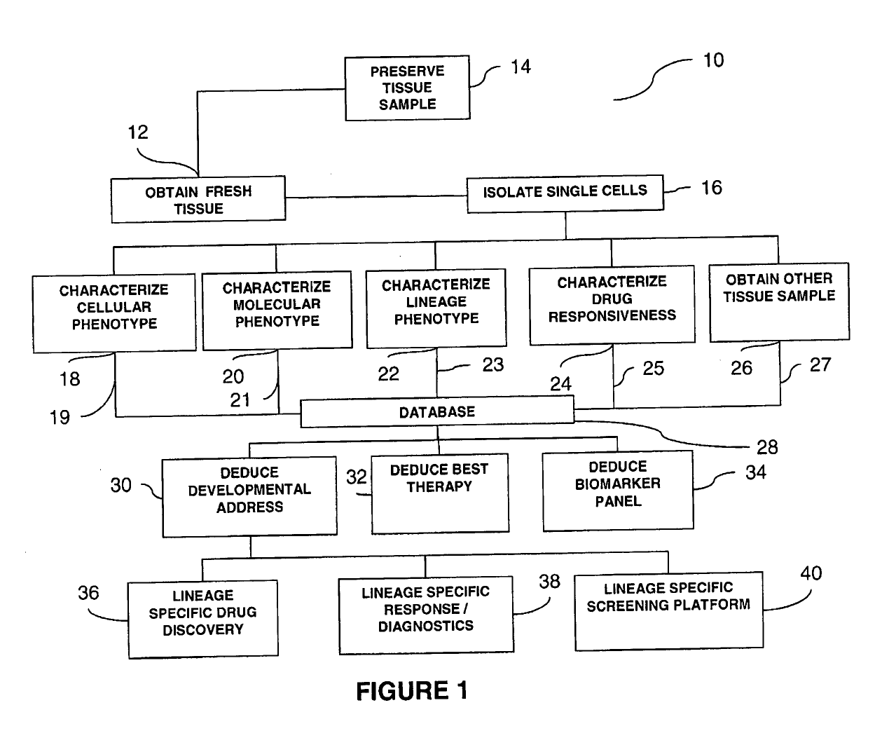

FIG. 1 is a flow diagram of one preferred process 10 of the invention. Referring to FIG. 1, and to the preferred embodiment depicted therein, in step 12 of such process, fresh tissue is obtained from a viable biological organism such, as, e.g., a human being. The tissue may be, e.g., tissue from a heart, lung, blood, liver, brain, hair, etc. The tissue may be normal tissue and/or abnormal tissue. As the term is used in this specification, the term tissue refers to an aggregate of cells and intercellular material that forms a definite structure in which the cells are generally of similar structure and function.

In one embodiment, the tissue is tissue from a malignant tumor. As is known to those skilled in the art, the term malignant is descriptive of tumor that metastasizes and endangers the life of an organism.

In another embodiment, the tissue is tissue that is not malignant but is otherwise abnormal. Thus, the tissue may be tissue infected with a virus or bacteria, or tissue that is malfunctioning (such as in, e.g., hypothyroidism), etc.

In yet another embodiment, the tissue is tissue that is neither malignant nor abnormal but is normal in every respect.

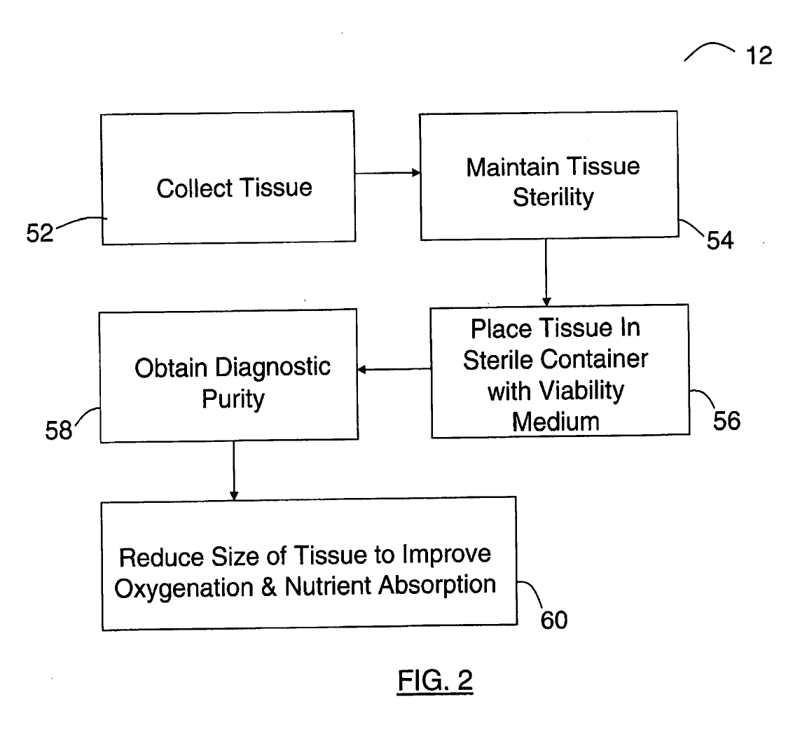

Referring again to step 12 in FIG. 1, in another embodiment, the tissue is obtained from one or more microorganisms such as, e.g., a bacterium, a fungus, a virus, etc. This step 12 is shown in greater detail in FIG. 2.

Referring to FIG. 2, and to the preferred embodiment depicted therein, one may collect the desired tissue by conventional means as shown in step 52. Thus, e.g., one may use one or more of the tissue collection methods disclosed in U.S. Pat. Nos. 5,624,418 (collection and separation device), 6,139,508 (articulated medical device), 6,036,698 (expanded ring percutaneous tissue removal device), 6,689,145 (apparatus for collecting and staging tissue), 6,702,831 (excisional biopsy devices and methods), 6,468,226 (remote tissue biopsy apparatus and associated methods), 6,022,362 (excisional biopsy devices and methods), 6,440,147 (excisional biopsy devices and methods), 5,782,764 (fiber composite invasive medical instruments), 4,966,162 (flexible endoscope assembly), 5,449,001 (biopsy needle), 6,471,709 (expandable ring percutaneous tissue removal device), 5,338,294 (urological evacuator), 5,290,303 (surgical cutting instrument), 5,275,609 (surgical cutting instrument), 5,183,052 (automatic biopsy instrument with cutting cannula), 5,569,284 (morecellator), 5,409,454 (apparatus for atherectomy), and the like. The entire disclosure of each of these United States patents is hereby incorporated by reference into this specification.

Referring to step 54 in FIG. 2, the desired tissue is collected, preferably in a sterile manner, and the sterility of the tissue so collected is maintained. As those who are knowledgeable in the art are aware, the term sterile means free from living germs or microorganisms. Thus, by way of illustration and not limitation, conventional sterile operating room procedures may often be used to insure sterile collection of the tissue from the patient's body. Reference may be had, e.g., to U.S. Pat. No. 6,322,533, “Apparatus for two-path distribution of a sterile operating fluid . . . .” The entire disclosure of this United States patent is hereby incorporated by reference into this specification.

In one preferred embodiment, and referring to step 56 of FIG. 2, after the desired tissue has been removed from the biological organism, it is placed in a sterile container along with a viability medium. The sterile container can be a conventional container, such as a test tube or a Petri dish and the like, that has undergone sterilization. Reference may be had, e.g., to U.S. Pat. Nos. 3,698,450 (sterile container filling mechanism), 3,715,047 (silicone stopper for sterile container), 3,941,245 (sterile container for enclosing a contaminated article), 3,988,873 (method for enclosing a contaminated article in a sterile container), 4,056,129 (closable sterile container) (, 4,124,141 (sterile container), 4,982,615 (sterile container for collecting biological samples for purposes of analysis), 5,178,278 (sterile container with tear-away throat), 5,462,526 (flexible sterile container), 5,492,243 (sterile container), 6,371,326 (sterile container for medical purposes), and the like. The entire disclosure of each of these United States patents is hereby incorporated by reference into this specification.

Referring again to FIG. 2, as those that are knowledgeable in the art are aware, sterilization is the complete destruction of all bacteria and other infectious organisms in an industrial, food, or medical product; it must be followed by aseptic packaging to prevent recontamination, usually by hermetic sealing. The sterilization can be accomplished through conventional methods involving either wet or dry heat, the use of chemicals such as formaldehyde and ethylene oxide filtration, and irradiation by UV or gamma radiation.

In one preferred embodiment, the desired tissue is placed in the sterile container within 3 hours of removal from the biological organism. In another preferred embodiment, the desired tissue is placed in the sterile container within about 1 hour of removal from the biological organism. In another preferred embodiment, the desired tissue is placed in the sterile container within about 15 minutes of removal from the biological organism.

Referring again to step 56 of FIG. 2, and in the preferred embodiment depicted therein, the desired tissue is placed in an enhanced viability medium which is comprised of a viability factor that, preferably, is essential for the cell's viability. As such term is used in this specification, the term “viability factor” refers to a factor that is required for the cell's viability and whose absence will lead to the cell's death. Reference may be had, e.g., to articles by O. S. Frankfurt et al. (“Protection from Apoptotic Cell Death by Inerleukin-4 is Increased in Lymphocytic Leukemia Patients,” Leuk. Res. 21:9-16, Elsevier Science, Ltd., January, 1997), by A. Horigome et al. (“Tacrolimus-inducted apoptosis and its prevention by interleukin blood mononuclear cells,” Immunopharmacology, 39:21-30, Elsevier Science B.V.), by H. Lindner et al. (“Peripheral Blood Mononuclear Cells Induce Programmed Cell Death . . . ,” Blood 89:1931-1938.

The viability factor may be a viability hormone such as, e.g., a stem cell viability factor. Reference may be had, e.g., to U.S. Pat. Nos. 5,601,056 (use of stem cell factor interleukin-6 . . . to induce the development of hematopoietic stem cells), 5,786,323 (use of stem cell factor and soluble interleukin-6 receptor to induce the development of hematopoietic stem cells), 5,861,315 (use of stem cell factor and soluble interleukin-6 receptor for the ex vivo expansion of hematopoietic multipotential cells), 5,885,962 (stem cell factor analog compositions), 6,824,973 (method of promoting stem cell proliferation or survival by contacting a cell with a stem cell factor-like polypeptide), 6,852,313 (method of stimulating growth of melanocyte cells by administering stem cell factor), and the like. The entire disclosure of each of these United States patents is hereby incorporated by reference into this specification.

By way of further illustration, the viability hormone may be erythropoietin. As is known to those skilled in the art, erythoropoietin is a glycoprotein mitogen and hormone with a molecular weight of about 23,000 Daltons that is produced by the kidneys and that stimulates the formation of erythrocytes; and its presence is essential for the viability of erythroid cells. Reference may be had, e.g., to U.S. Pat. Nos. 5,830,851 (methods of administering peptides that bind to the erythorpoietini receptor), 5,986,047 (peptides that bind to the erythropoietin receptor), 6,531,121 (protection and enhancement of erythropoietin-responsive cells, tissues, and organs), and the like. The entire disclosure of each of these United States patents is hereby incorporated by reference into this specification.

By way of further illustration, the viability hormone may be a follicle stimulating hormone. As is known to those skilled in the art, follicle stimulating hormone is the gonadotropic protein hormone, secreted by the anterior lobe of the pituitary gland, that stimulates the growth of ovarian follicles and the secretion of estadiol in the female and spermatogenesis in the male; its presence is essential for the viability of ovarian follicular cells. Reference may be had, e.g., to U.S. Pat. Nos. 5,744,448 (human follicle (human follicle stimulating hormone receptor), 5,767,067 (follicle stimulating hormone), 6,306,654 (follicle stimulating hormone-glyosylation analogs), 6,737,515 (follicle stimulating hormone-glycosylation analogs), and the like The entire disclosure of each of these United States patents is hereby incorporated by reference into this specification.

By way of yet further illustration, the viability hormone may be a melanocyte stimulating hormone. As is known to those skilled in the art, such a hormone is one of two peptide hormones, denoted alpha and beta, that are produced by the posterior lobe of the pituitary gland and that have a darkening effect by causing the dispersion of melanin pigments in the melanocytes. Reference may be had, e.g., to U.S. Pat. Nos. 5,126,327 (melanocyte-stimulating hormone inhibitor), 5,849,871 (alpha-melanocyte stimulating hormone receptor), 6,268,221 (melanocyte stimulating hormone receptor), 6,660,856 (antagonists of alpha-melanocyte stimulating hormone and methods based thereon), and the like. The entire disclosure of each of these United States patents is hereby incorporated by reference into this specification.

By way of yet further illustration, the viability hormone may be thyrotropin. As is known to those skilled in the art, thyrotropin is a protein hormone, secreted by the anterior lobe of the pituitary gland, that stimulates the synthesis of thyroid hormones and the release of thyroxine by the thyroid gland; and its presence is essential for the viability of thyroid epithelial cells Reference may be had, e.g., U.S. Pat. Nos. 3,959,248 (analogs of thyrotropin-releasing hormone), 4,493,828 (use of thyrotropin releasing hormone and related peptides as poultry growth promotants), 5,864,420 (thyrotropin-releasing hormone analogs), 5,879,896 (method of screening for inhibitors of human thyrotropin releasing hormone receptor), 6,441,133 (thyrotropin-releasing hormone receptor), and the like. The entire disclosure of each of these United States patents is hereby incorporated by reference into this specification.

By way of yet further illustration, the viability hormone may be epidermal growth factor. As is known to those skilled in the art, epidermal growth factor is a polypeptide mitogen, with a molecular weight of about 6400, that stimulates the proliferation of epidermal and epithelial tissues and the presence of which is required for the viability of such tissues. Reference may be had, e.g., to U.S. Pat. Nos. 5,960,820 (epidermal growth factor receptor targeted molecules), 6,129,915 (epidermal growth factor receptor antibodies), 6,255,452 (epidermal growth factor inhibitor), and the like. The entire disclosure of each of these United States patents is hereby incorporated by reference into this specification.

As will be apparent, one can determine by conventional means whether a particular factor, such as, e.g., a prospective viability hormone, is indeed essential for the survival of a particular cell by testing the viability of such cell in both the presence of and the absence of such factor. Reference may be had, e.g., to U.S. Pat. No. 6,843,980, that describes “Methods for using annexin for detecting cell death in vivo and treating associated conditions.” Reference also may be had to U.S. Pat. Nos. 5,185,450 (tetrazolium compounds for cell viability assays), 5,314,805 (dual-fluorescence cell viability assay using ethidium homodimeer and calcein AM), 6,403,378 (cell viability assay reagent), and the like. The entire disclosure of each of these United States patents is hereby incorporated by reference into this specification.

The disclosure contained in U.S. Pat. No. 6,403,378 is of interest. As is disclosed in this patent, “Two dyes are generally used to stain cells in a suspension for viability analysis. One dye consists of a membrane permeant DNA dye that labels all intact cells in a suspension, whereby they emit light at one wavelength. A non-permeant DNA dye labels all dead cells.”

U.S. Pat. No. 6,403,378 also discloses that “In one method of analysis, the cells in the cell suspensions are stained and a traditional hemacytometer is used to differentiate the cells. Another analysis system utilizes dual-color fluorescence in combination with forward light scatter to determine the concentration of nucleated cells and cell viability. Cells are analyzed by providing relative movement between the sample suspension containing the cells and an excitation light beam, whereby labeled cells pass through the light beam and emit light at a wavelength characteristic of the permeant and non-permeant dye. The detection system includes filters and detectors which detect the light emitted at the two wavelengths. The cells also scatter light, whereby all particles in the sample suspension are detected. Once a cell has been detected on the permeant dye channel, the light scatter profile is evaluated to assure that the cell is of sufficient size to be an intact cell and not simply a free nucleus or other cell fragment. The second dye permeates all cells with damaged or “leaky” membranes. The dye emits fluorescent light at a different wavelength range than that of the cells stained with permeant dye. In this manner all cells are detected by detecting the light emitted by the second dye at one wavelength, and non-viable cells are detected by detecting light emitted by the permeant dye at the other wavelength. Thus, an absolute count of cells and percent viability can be obtained from the data.”

U.S. Pat. No. 6,403,378 also discloses that “To obtain reliable results for different cell concentrations, using a two-dye method it is necessary to carefully control the amount of each of the dyes used to stain or tag the cells. This is a time-consuming procedure and may lead to variability in results obtained.”

As will be apparent to those skilled in the art, when a particular hormone is found to be essential for the viability of a particular cell, it is deemed to be a cell type specific viability hormone.

Referring again to FIG. 2, and in step 56 thereof, one may use other known means for insuring viability. Various media for maintaining viability are well known to those skilled in the art. Reference may be had, e.g., to U.S. Pat. No. 5,543,316, for an “Injectable culture medium for maintaining viability of myoblast cells.” The entire disclosure of such United States patent is hereby incorporated by reference into this specification.

In one embodiment, the base viability medium is be a sterile saline solution, or a balanced salt solution, or a glucose containing culture medium, serum, or the like.

Referring again to FIG. 2, and in step 56 thereof, the cell type specific hormone preferably is present in the viability medium at a concentration from about 0.01 to about 10 micrograms per milliliter; in one aspect of this embodiment, the hormone is present in a range of from about 0.1 to about 5 micrograms per milliliter. In yet another embodiment, the hormone is present at a concentration of from about 0.3 to about 3 micrograms per milliliter.

In one preferred embodiment, the desired tissue is maintained with at least about 90% viability. In another preferred embodiment, the desired tissue is maintained with at least about 95% viability. In another preferred embodiment, the desired tissue is maintained with at least about 99% viability. The desired tissue is preferably tested for viability using the tryphan blue exclusion test as is described in U.S. Pat. Nos. 5,739,274 (active component of parathyroid hypertensive factor), 6,008,007 (radiation resistance assay for predicting treatment response and clinical outcome), 6,261,795 (radiation resistance assay for predicting treatment response and clinical outcome), 6,447,810 (composition of multipurpose high functional alkaline solution composition, preparation thereof, and for the use of nonspecific immunostimulator), 6,673,375 (composition of multipurpose high functional alkaline solution composition, preparation thereof, and for the use of nonspecific immunostimulator), and 6,699,851 (cytotoxic compounds and their use). The entire disclosure of these United States patents are hereby incorporated by reference.

Referring to step 58 in FIG. 2, in one preferred embodiment, the desired tissue is processed to obtain a diagnostic purity. As is known by those skilled in the art, diagnostic purity refers to characterizing the cells that are purified from the surgical tissue (containing the tumor and some normal tissue) and at least 90 percent of the cells are the same as the original diagnosis. As is known to those skilled in the art, one may determine purity by visual observation of morphology under a microscope. In one preferred embodiment, the desired cells are tumor cells and not the surrounding normal cells. In one preferred embodiment, a diagnostic purity of at least about 90 percent is obtained. In another preferred embodiment, a diagnostic purity of at least about 95 percent is obtained. In another preferred embodiment, a diagnostic purity of at least about 99 percent is obtained. This diagnostic purity is preferably obtained by separating the desired tissue from the surrounding tissue. In one preferred embodiment, the desired tissue is a tumor and the surrounding tissue is normal.

Referring again to step 58 in FIG. 2, in one preferred embodiment, the purity of the desired tissue can be measured by conventional means such as one or more of the processes described in U.S. Pat. Nos. 5,741,648 (cell analysis method using quantitative fluorescence image analysis) and 5,733,721 (cell analysis method using quantitative fluorescence image analysis); the entire disclosure of each of these United States patents is hereby incorporated by reference into this specification.

U.S. Pat. No. 5,733,721 describes a quantitative fluorescence image analysis (QFIA) method and claims (in claim 1): “A method of analyzing a cell sample derived from urine or from a bladder wash, comprising: providing a prepared slide, the prepared slide having been prepared by applying a portion of a cell sample to a slide, the portion of the cell sample treated with a fixative composition comprising a salt of ethylenediaminetetraacetic acid effective in inhibiting formation of substantially all of the crystals in the cell sample prior to application of the portion of the cell sample to the slide leaving the prepared slide substantially free of crystals for improving microscopic analysis of the cell on the prepared slide, then treating the slide with a fluorescent label for labeling the cytological marker to form a labeled cytological marker; irradiating a portion of the prepared slide with an amount of an excitation wavelength of light effective in causing the fluorescent label in a cell to emit fluorescent light having an emission wavelength for forming a field image; using a microscope means to select cell images on the field image; obtaining a number related to the selected cell images; and outputting the number for use in classifying the cell sample.” U.S. Pat. No. 5,733,721 further claims (in claim 23): “A method of analyzing a cell sample derived from urine or from a bladder wash, comprising: providing a prepared slide, the prepared slide having been prepared by applying a portion of a cell sample to a slide, the portion of the cell sample treated with a fixative composition comprising a salt of ethylenediaminetetraacetic acid effective in inhibiting formation of substantially all of the crystals in the cell sample prior to application of the portion of the cell sample to the slide leaving the prepared slide substantially free of crystals for improving microscopic analysis of the cell on the prepared slide, then treating the slide with a first fluorescent label for labeling the first cytological marker to form a labeled first cytological marker and the second fluorescent label for labeling the second cytological marker to form a labeled second cytological marker; irradiating a first portion of the prepared slide with an amount of a first excitation wavelength of light effective in causing the first fluorescent label in the cell to emit fluorescent light having a first emission wavelength for forming a first field image; using a microscope means to select first cell images on the first field image; obtaining a first number related to the selected first cell images; irradiating the second portion of the prepared slide with a second excitation wavelength of light effective in causing the second fluorescent label to emit fluorescent light having a second emission wavelength for forming a second field image wherein the second portion may be the same as the first portion; using the microscope means to select second cell images on the second field image; obtaining a second number related to the selected second cell images; and outputting the first number and the second number for use in classifying the cell sample.”

Referring to step 60 in FIG. 2, in one preferred embodiment, the desired tissue is separated into smaller pieces to allow for oxygenation of the cells of the tissue and to allow for nutrient absorption by the cells of the tissue. In one preferred embodiment, the desired tissue is sliced into thin slices of preferably from about 2 millimeters thickness. In another preferred embodiment, the desired tissue is sliced into thin slices of preferably from about 0.50 millimeters thickness or less. In another preferred embodiment, the desired tissue is sliced into thin slices of from about 0.01 millimeter or less. One may use conventional means to disaggregate the tissue sample. Reference may be had, e.g., to U.S. Pat. No. 3,941,317, for a “Method and apparatus for tissue disaggregation.” The entire disclosure of this United States patent is hereby incorporated by reference into this specification.

Referring again to FIG. 1, after the desired tissue sample is obtained in step 12, then it may either be preserved (in step 14), and/or isolated single cells may be obtained in step 16. One may isolate single cells by conventional means such as, e.g., preparing single cell suspensions. Reference may be had to U.S. Pat. Nos. 4,350,768 (method for preparing single cell suspension), 4,413,059 (apparatus for preparing single cell suspension), 5,728,580 (method for inducing single cell suspension in insect cell lines), 5,744,363 (method for establishing a tumor cell line by preparing single cell suspension of tumor cells from tumor biopsies), 6,103,526 (Spodoptera frugiperda single cell suspension cell line in serum-free media), and the like. The entire disclosure of each of these United States patents is hereby incorporated by reference into this specification.

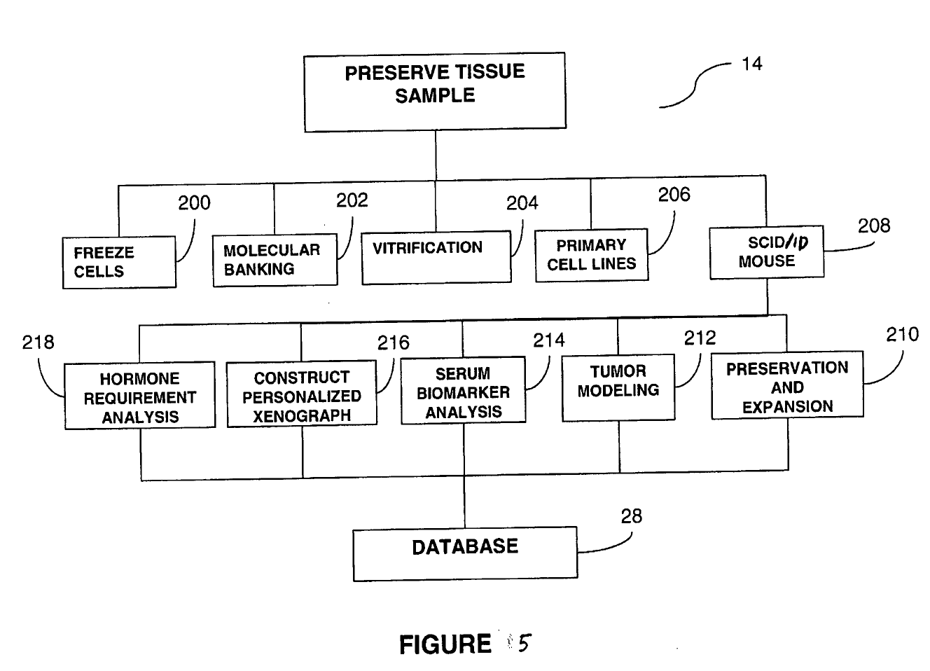

Referring to FIG. 1, and to the preferred embodiment depicted therein, in step 14 of such process, the desired tissue is preserved in a viable state and the tissue viability is preferably tested using the tryphan blue exclusion test. By way of illustration, an example of a method of tissue preservation is claimed in U.S. Pat. No. 6,569,615 (composition and methods for tissue preservation); the entire disclosure of this United States patent is hereby incorporated by reference. One preferred means of preserving such tissue and/or cells will be discussed elsewhere in this specification with reference to FIG. 5.

Referring to step 16 in FIG. 1, and to the preferred embodiment depicted therein, in one preferred embodiment, the cells are obtained by one or more of the processes described in U.S. Pat. Nos. 5,733,721 (cell analysis method using quantitative fluorescence image analysis), 5,741,648 (cell analysis method using quantitative fluorescence image analysis), 5,824,495 (cell fixative and preparation, kit and method) 6,194,165 (cell fixative and preparation composition, kit and method), and 6,372,450 (method of treating cells); the entire disclosure of each of these United States patents is hereby incorporated by reference into this specification.

In such step 16, certain single cells are isolated. The single cells may be obtained by conventional means. To this end, one may use one or more of the processes claimed in U.S. Pat. Nos. 5,827,735 (pluripotent mesenchymal stem cells), 6,420,105 (method for analyzing molecular expression of function in an intact single cell), 6,541,247 (method for isolating ependymal neural stem cells), 6,686,197 (method for producing preparations of mature and immature pancreatic endocrine cells), and the like. The entire disclosure of each of these United States patents is hereby incorporated by reference into this specification.

By way of further illustration, and as is claimed in U.S. Pat. No. 6,077,684, one may conduct the step of “isolating a single cell suspension from the sample.” Thus, e.g., and as is disclosed in columns 14 and 15 of such patent, “Prior to any chemotherapy, a sample of venous blood (e.g., 1-30 ml) or a sample of bone marrow (e.g., 2-20 ml) is obtained by direct needle aspiration under sterile conditions. The samples are drawn into a heparinized syringe and diluted with RPMI-1640 medium that contains no phenol red. The mononuclear fraction of each sample is isolated by centrifugation using Ficoll-Hypaque. If erythrocytes contaminate the mononuclear cell fraction, then they are removed by treatment with red cell lysis buffer. After washing three times in phosphate buffered saline, an aliquot of the mononuclear cells is analyzed by either light microscopy or flow cytometry for purity and viability. The specific MAb's that recognized the leukemia cells in the diagnostic testing are used to check purity while 7-amino-actinomycin D (7AAD) is used to check viability. If purity and viability are both greater than 90%, then the cells are aliquoted for the present assays and for cryopreservation in RPMI-1640 containing 20% fetal bovine serum and 10% dimethylsulfoxide. Greater than 90% purity and viability would be expected in most cases with a high leukemic cell count in either the blood or bone marrow. If the mononuclear cell fraction purity is less than 90%, then the cells are further purified. T-lymphocytes and monocytes are removed by negative selection using immunomagnetic separation. MAb's to CD2 for T-cell removal and CD14 for monocyte removal and Dynabeads (Dynal, Inc.) are used in those cases in which the diagnostic immunophenotyping shows that the leukemic cells lack these surface antigens. After these immunomagnetic separations, the leukemic cell population will again be tested for purity.

In one embodiment, the tissue is preferably rendered into smaller pieces and then digested with a series of enzymes (such as, e.g., trypsin, collagenase, lipase, and the like) to disaggregate the tissue into stromal cell, connective tissue, and tumor cells such that the tumor cells can then be readily isolated.

As the term is used in this specification, the term tissue refers to an aggregate of cells and intercellular material that forms a definite structure in which the cells are generally of similar structure and function.

As is known to those skilled in the art, digestion involves the chemical or enzymatic hydrolysis of macromolecules. Reference may be had, e.g., U.S. Pat. Nos. 4,350,768 (method for preparing single cell suspensions), 4,413,059 (apparatus for preparing single cell suspensions), 5,728,580 (methods and culture media for inducing single cell suspension in insect cell lines), and 6,420,105 (method for analyzing molecular expression or function in an intact single cell), to descriptions of the enzymatic preparation of single cell suspensions. The patents also refer to mechanical methods of preparing single cell suspensions. The disclosures of these patents are hereby incorporated by reference.

If trypsinization is used, the cells must recover the functionality of the membrane proteins.

In one preferred embodiment, and referring to step 16 of FIG. 1, during the time that the single cells are isolated, it is preferred to expose such cells to an oxygen-containing atmosphere containing at least 1 volume percent of oxygen and, more preferably, at least about 5 volume percent of oxygen. In one embodiment, the cells are maintained in an atmosphere of at least about 10 percent oxygen. In another embodiment, the cells are maintained in an atmosphere of at least about 20 percent oxygen. In one aspect of this embodiment, either oxygen and/or an oxygen-containing gas (such as a mixture of 5 volume percent carbon dioxide and 95 volume percent of oxygen) is bubbled through a cell solution medium comprised of the cells in question to adequately oxygenate substantially all of the cells in the medium.

Referring again to FIG. 1, it is preferred, while conducting the cell isolation step 16, to maintain the cells within a temperature of from about 22 to about 39 degrees Celsius.

It is also preferred, while isolating the single cells in step 16, to continue to contact such cells with the enhanced viability medium introduced in step 12.

In one preferred embodiment, the single cells are isolated from a medium that contains a molecule that tends to prevent apoptosis. As known to those skilled in the art, apoptosis is one of the two mechanisms by which cell death occurs (the other being the pathological process of necrosis). Apoptosis is the mechanism responsible for the physiological deletion of cells, and is characterized by distinctive morphologic changes in the nucleus and cytoplasm, chromatin cleavage at regularly spaced sites, and the endonucleolytic cleavage of genomic DNA at internucleosomal sites. Apoptosis serves as a balance to mitosis in regulating the size of animal tissues and in mediating pathologic processes associated with tumor growth. These molecules that prevent apoptosis are well known and are described, e.g., in an article by H Rui et al., “Activation of the Jak20Stat5 signaling pathway in Nb2 lymphoma cells by an anti-apoptoic agent, aurintricarboxylic acid,” J. Biol. Chem. Jan. 2, 1998; 273 (1):28-32.

Aurintricarboxylic acid is well known and is described, e.g., in U.S. Pat. No. 5,431,185, the entire disclosure of which is hereby incorporated by reference into this specification. As is disclosed in such patent, U.S. Pat. No. 4,007,270 to Bernstein et al. discloses that aurintricarboxylic acid (ATA) and certain of its derivatives and salts are useful as complement inhibitors which play an important role as mediators in immune, allergic, immunochemical and immunopathological reactions. As is well known in the field, the term “complement” refers to a complex group of proteins in body fluids that, working together with antibodies or other factors, play an important role as mediators of immune, allergic, immunochemical and/or immunopathological reactions. The reactions in which complement participates take place in blood serum or in other body fluids, and hence are considered to be humoral reactions. Aurins (free acid and ammonium salt) may be prepared according to the method of G. B. Heisig and W. M. Lauer, Org. Syn. Coll. Vol. 1 (second Ed.), 54-55 (1932); Holaday, D. A., J. Am. Chem. Soc., 62, 989 (1940); The Merck Index, 8th Ed. (1968), page 42; and Caro, Ber., 25, 939 (1892). Esterification with an alcohol and acylation in the presence of an acid provide the derivatives of this invention. The salts of the free acid and acylates may be obtained by treatment thereof with a suitable base in an aqueous alcohol. The patent discloses a method of inhibiting the complement system in blood serum subjecting the serum to aurintricarboxylic acid or its derivatives or salts and that ATA has anti-inflammatory properties. The patent discloses Aurintricarboxylic acid (ATA) is a heterogeneous mixture of polymers that forms when salicylic acid is treated with formaldehyde, sulfuric acid and sodium nitrite (see Cushman, M. et al. “Preparation and Anti-HIV Activities of Aurintricarboxylic Acid Fractions and Analogues: Direct Correlation of Antiviral Potency with Molecular Weight”, J. Med. Chem., Volume 34, (1991) pp. 329-337; Cushman, M. et al. “Synthesis and Anti-HIV Activities of Low Molecular Weight Aurintricarboxylic Acid Fragments and Related Compounds”, J. Med. Chem., Volume 34, (1991) pp. 337-342).

In one embodiment, and referring again to step 16 of FIG. 1, the cells are isolated in step 16 while in the presence of an anti-apoptic agent such as, e.g., aurintricarboxylic acid (ATA) and optionally, other agents that promote cell viability. Aurintricarboxylic acid is known to cause cell death, in appropriate concentrations. Thus, e.g., U.S. Pat. No. 5,434,185 describes in claim 1 “1. A method for inhibiting angiogenesis in an animal comprising administering an effective amount to inhibit angiogenesis of aurintricarboxylic acid, its analogues, or salts to said animal.” Claim 3 of this patent describes “3. A method according to claim 1, wherein said effective amount comprises about 10 mg/kg body weight of the host aurintricarboxylic acid.” In one preferred embodiment, not shown, the dosage of aurintricarboxylic acid that is known to cause cell death is from 0.01 micromoles to 0.1 micromoles. Thus the aurintricarboxylic acid needs to be applied in doses not approaching this level.

Regardless of the process of isolation of the single cells used in step 16, it is preferred that the single cells so isolated have a viability of at least about 90 percent and a purity of at least about 90 percent. In one embodiment, the viability and the purity is at least about 95 percent. One may determine the viability of the cell samples by tryphan blue exclusion. One may determine purity by visual observation of morphology under a microscope.

Referring again to FIG. 1, and in the preferred embodiment depicted therein, the single cells isolated in step 16 of the process 10 may be used to characterize the cellular phenotype (in step 18), and/or to characterize the molecular phenotype of the cell (in step 20), and/or to characterize the lineage phenotype of the cell (in step 22), and/or to characterize the drug response of the cell (in step 24). Alternatively, or additionally, one may also obtain patient samples for additional analyses or information.

Referring again to FIG. 1, and to step 18 of process 10, the cellular phenotype of the isolated single cells is characterized. As is known to those skilled in the art, the term phenotype refers to the physical appearance and the observable properties of an organism that are produced by the interaction of the genotype with the environment. The cellular phenotype refers to the physical appearance and the observable properties of a cell that are produced by the expression of specific sets of genes and proteins.

One may characterize the cellular phenotype of the isolated single cells by conventional means. Reference may be had, e.g., to U.S. Pat. Nos. 6,197,523 (method for the detection, identification, enumeration, and confirmation of circulating cancer and/or hematologic progenitor cells in whole blood), 5,496,704 (in vitro detection of formed elements in biological samples), 5,403,714 (method for in vitro detection of formed elements in biological samples), 4,099,917 (process for preparing a cell suspension from blood for discrimination of white blood cells and platelets from other blood particles), and the like. The entire disclosure of each of these United States patents is hereby incorporated by reference into this specification.

As will be apparent, the characterization of the cellular phenotype of the isolated single cells furnish some gross information about the broad lineage of the isolated single cells, i.e., whether such cells are brain cells, breast cells, lung cells, pancreas cells, etc.

Referring again to FIG. 1, and in step 20 thereof, the molecular phenotype of the isolated single cells is characterized. This will furnish more information regarding the broad lineage of the isolated single cells, i.e., whether the single cells are expressing genes and proteins from brain tissue, breast tissue, lung tissue, etc.

One may characterize the molecular phenotype of the isolated single cell populations by conventional means. Thus, e.g., one may use the characterization processes described in U.S. Pat. Nos. 6,406,630 (treating cancers associated with overexpression of HER-2/neu), 6,291,496 (treating cancers associated with overexpression of class I family . . . ), 6,200,760 (method of screening agents as candidates for drugs or sources of drugs), 5,876,932 (method for gene expression analysis), 6,203,987 (methods for using co-regulated genesets to enhance detection and classification of gene expression patterns), 6,406,921 (protein arrays for high-throughput screening), 6,355,423 (methods and devices for measuring differential gene expression), 6,475,809 (protein arrays for high-throughput screening), 6,537,749 (addressable protein arrays), 6,548,021 (surface-bound, double-stranded DNA protein arrays), 6,635,423 (informative nucleic acid arrays), 6,618,679 (methods for analysis of gene expression), 6,653,135 (dynamic protein signature assay), 6,696,620 (immunoglobulin binding protein arrays), and the like. The entire disclosure of each of these United States patents is hereby incorporated by reference into this specification.

In one embodiment, and referring again to step 20 of FIG. 1, the molecular phenotype is characterized by the process described in U.S. Pat. No. 6,221,600 (combinatorial oligonucleotide PCR: a method for rapid, global expression analysis), the entire disclosure of which is hereby incorporated by reference into this specification. This patent claims: “A method comprising: a) obtaining a DNA comprising an anchorable moiety; b) cleaving said DNA with a first restriction endonuclease; c) ligating a linker molecule to cleaved DNA produced in step b;d) immobilizing linker ligated DNA through said anchorable moiety; e) cleaving DNA immobilized in step d with a second restriction endonuclease; f) ligating a second linker molecule to DNA cleaved in step e; g) amplifying DNA ligated in step f.” As is disclosed in the abstract of this patent, “The present invention relates to a method for the detection of gene expression and analysis of both known and unknown genes. The invention is a highly sensitive, rapid and cost-effective means of monitoring gene expression, as well as for the analysis and quantitation of changes in gene expression for a defined set of genes and in response to a wide variety of events. It is an important feature of the present invention that no single molecular species of cDNA gives rise to more than one fragment in the collection of products which are subsequently amplified and representative of each expressed gene. This achievement is facilitated by immobilizing the cDNA prior to digesting and then digesting with sequentially with two frequently cutting enzymes. Linker oligomers are ligated to each cut site following the respective digestion. Primers, complementary to the oligomer sequence with an additional 3′ variable sequence are used to amplify the fragments. Using an array of fragments theoretically facilitates the amplification of all of the possible messages in a given sample.”

Referring again to FIG. 1, and in the preferred embodiment depicted therein, in step 22 of process 10 the lineage phenotype of the isolated single cells is characterized. As is known to those skilled in the art, the lineage of a cell is its developmental pathway. Development, as used in this specification, refers to the series of orderly changes by which a mature cell, tissue, organ, organ system, or organism comes into existence. Each cell is part of a developmental pathway that, through a process of differentiation, proliferation, and maturation, produces functional cells from non-functional stem or seed cells.

Thus, e.g., and as is disclosed in U.S. Pat. No. 6,248,587 (the entire disclosure of which is hereby incorporated by reference into this specification), “Mesenchymal stem cells (MSC) are pluripotent progenitor cells that possess the ability to differentiate into a variety of mesenchymal tissue, including bone, cartilage, tendon, muscle, marrow stroma, fat and dermis as demonstrated in a number of organisms, including humans (Bruder, et al., J. Cellul. Biochem. 56:283-294 (1994). The formation of mesenchymal tissues is known as the mesengenic process, which continues throughout life, but proceeds much more slowly in the adult than in the embryo (Caplan, Clinics in Plastic Surgery 21:429-435 (1994). The mesengenic process in the adult is a repair process but involves the same cellular events that occur during embryonic development (Reviewed in Caplan, 1994, supra). During repair processes, chemoattraction brings MSC to the site of repair where they proliferate into a mass of cells that spans the break. These cells then undergo commitment and enter into a specific lineage pathway (differentiation), where they remain capable of proliferating. Eventually, the cells in the different pathways terminally differentiate (and are no longer able to proliferate) and combine to form the appropriate skeletal tissue, in a process controlled by the local concentration of tissue-specific cytokines and growth factors (Caplan, 1994, supra).”

Referring again to FIG. 1, in step 22 the lineage pathway of the isolated single cells is determined. This can be accomplished by conventional means such as, e.g., the processes disclosed in U.S. Pat. Nos. 5,817,773 (stimulation, production, culturing and transplantation of stem cells by fibroblast growth factors), 6,248,547 (process for promoting lineage-specific cell proliferation and differentiation), 6,268,212 (tissue specific transgene expression), 6,280,724 (composition and method for preserving progenitor cells), 6,380,458 (cell-lineage specific expression in transgenic zebrafish), 6,391,297 (differentiation of adipose stromal cells in osteoblasts), 6,548,299 (lymphoid-tissue specific cell production from hematopoietic progenitor cells in three-dimensional devices), and the like. The entire disclosure of each of these United States patents is hereby incorporated by reference into this specification.

In one preferred embodiment, the lineages of the isolated single cells are analyzed to determine, e.g., the presence of known proteins and/or antigens associated with specific lineages of such cells. By way of illustration, these may include hormone receptors, lineage specific kinases, lineage specific transcription factors and/or regulators, lineage specific gene rearrangements, and the like.

Referring again to FIG. 1, in step 24 of process 10, the drug response of the isolated single cells is characterized. One may use conventional means for determining the drug response of such cells such as, e.g., the means disclosed in U.S. Pat. Nos. 4,816,395 (method for predicting chemosensitivity of anti-cancer drugs), 4,937,182 (method for predicting chemosensitivity of anti-cancer drugs), 6,468,547 (enhancement of tumor cell chemosensitivity), 6,521,407 (methods for determining chemosensitivity of cancer cells based on expression of negative and positive signal transduction factors), 6,620,403 (in vivo chemosensitivity screen for human tumors), and the like. The entire disclosure of each of these United States patents is hereby incorporated by reference into this specification.

In one embodiment, the process of U.S. Pat. No. 6,258,553 of Kravtsov is used to effectuate step 24. Claim 1 of this patent describes: “A method of determining the apoptosis-inducing activity of a substance, which comprises: a) measuring the optical density of a first cell culture at more than one time point, wherein the first cell culture was contacted with the substance; b) measuring the optical density of a second cell culture at more than one time point, wherein the second cell culture was not contacted with the substance; and c) determining a net slope, which is the difference between the optical density change over time of the first cell culture and the optical density change over time of the second cell culture; wherein a positive net slope indicates apoptosis-inducing activity of the substance.” The entire disclosure of this United States patent is hereby incorporated by reference into this specification.

Several patents and patent publications have been issued or published in the name of Vladimir D. Kravtsov. These include U.S. Pat. Nos. 6,077,684 and 6,258,553, International publications numbers WO 02/40702A2, WO 02/42749 A2, WO 02/46750 A2, WO 02/46751 A2, and Australian patent publications AU0225874A5, AU0239491A5, and AU0239771A5. The entire disclosure of each of these patents and patent applications is hereby incorporated by reference into this specification.

U.S. Pat. No. 6,077,684 is illustrative of some of the Kravtsov technology. This patent, in claim 1 thereof, describes: “A method of determining the anti-leukemic activity of a substance, comprising: a. obtaining a sample of cells from a subject with leukemia; b. isolating a single cell suspension from the sample; c. enriching the sample for leukemic cells by removing non-leukemic cells from the sample; d. placing the enriched leukemic cells in culture; e. exposing a culture of the enriched cells to the substance; f. incubating the cultured cells; g. measuring in a serial manner the optical densities of the culture exposed to the substance; h. measuring in a serial manner the optical densities of a culture of the enriched cells not exposed to the substance; i. subtracting at each serial time point the optical densities of the culture of cells not exposed to the substance from the optical densities of the culture of cells exposed to the substance, so as to obtain a net slope of the serially measured optical densities due to the apoptosis-inducing activity of the substance; j. correlating the slope of a net increase over time in the serially measured optical densities of the cells exposed to the substance with anti-leukemic activity” (see claim 1). As will be apparent, claim 1 of U.S. Pat. No. 6,077,684 describes a process for determining the sensitivity of anti-leukemic agents on leukemia cells.

By comparison, claim 2 of U.S. Pat. No. 6,077,684 describes a process for determining the resistance of leukemia cells to anti-leukemic agents. This claim discloses: “2. A method of determining resistance of leukemic cells to an anti-leukemic substance, comprising: a. obtaining a sample of cells from a subject with leukemia; b. isolating a single cell suspension from the sample; c. enriching the sample for leukemic cells by removing non-leukemic cells from the sample; d. placing the enriched leukemia cells in culture; e. exposing a culture of enriched cells to the substance; f. incubating the cultured cells; g. measuring in a serial manner the optical densities of the culture of enriched cells exposed to the substance; h. measuring in a serial manner the optical densities of a culture of the enriched cells not exposed to the substance; i. subtracting at each serial time point the optical densities of the culture of cells not exposed to the substance from the optical densities of the culture of cells exposed to the substance, so as to obtain a net slope of the serially measured optical densities due to the apoptosis-inducing activity of the substance; j. correlating the absence of a net increase or the presence of a reduced slope of a net increase over time in the optical densities of the culture exposed to the substance with resistance to the substance.”

By way of further comparison, claim 3 of U.S. Pat. No. 6,077,684 describes a process for determining the relative activity of anti-leukemic agents on leukemia cells. This claim describes: “3. A method of determining the relative potential effectiveness of a substance for use in anti-leukemic therapy for a selected subject having leukemia, comprising: a. obtaining a sample of cells from the subject with leukemia; b. isolating a single cell suspension from the sample; c. enriching the sample for leukemic cells by removing non-leukemic cells from the sample; d. placing the enriched leukemic cells in culture; e. exposing a culture of the enriched cells to a first selected substance or mixture of the first selected substance and other substances; f. exposing a culture of the enriched cells to a second selected substance or mixture of the second selected substance and other substances; g. incubating the cultured cells; h. measuring in a serial manner the optical densities of the cultures of enriched cells exposed to the first and second substances or mixtures of substances; i. measuring in a serial manner the optical densities of a culture of the enriched cells not exposed to a substance; j. subtracting at each serial time point the serially measured optical densities of the culture of cells not exposed to the substance from the optical densities of the culture of cells exposed to the first substance or mixture of substances and the optical densities of the culture of cells exposed to the second substance or mixture of substances, so as to detect differences in the net slopes of the serial optical densities due to differences in the apoptosis-inducing activity of the first and second substances or mixtures of substances; k. correlating the greater slope of a net increase over time in the serial optical densities of the culture of cells exposed to the first substance compared to the slope of a net increase over time in the serial optical densities of the culture of cells exposed to the second substance with the greater potential effectiveness of the first substance or mixture of the first substance and other substances in anti-leukemic therapy.”

The claims of U.S. Pat. No. 6,077,684 are limited to processes involving anti-leukemic agents. By comparison, the claims of U.S. Pat. No. 6,258,553 relate to agents that induce apoptosis. Claim 1 of this patent describes: “A method of determining the apoptosis-inducing activity of a substance, which comprises: a) measuring the optical density of a first cell culture at more than one time point, wherein the first cell culture was contacted with the substance; b) measuring the optical density of a second cell culture at more than one time point, wherein the second cell culture was not contacted with the substance; and c) determining a net slope, which is the difference between the optical density change over time of the first cell culture and the optical density change over time of the second cell culture; wherein a positive net slope indicates apoptosis-inducing activity of the substance.”

The process described in the Kravtsov patent publications is not adapted to either detect or diagnose or prepare therapy for or to monitor clonal cell populations. It is an object of one embodiment of this invention to provide a process for detecting, diagnosing, and preparing therapy for clonal cell populations.

Referring again to FIG. 1, and in step 26 thereof, one or more samples are obtained from a biological organism. This step 26 may be conducted at the time steps 12 and/or 14 are conducted, or thereafter, or before.

The additional material collected from the biological organism in step 26 may be, e.g., serum, cells that are not diseased (such as, e.g., somatic cells, lymphocytes, granulocytes, dendritic cells, and cytotoxic T lymphocytes (CTL)), and the like. In one particular embodiment, lymphocytes, granulocytes, dendritic cells, and CTLs are collected from the peripheral blood of an individual patient and are used as controls for toxicity and chemosensitivity testing of an individual patient's normal cells, e.g. “non-cancerous” cells, to assess the risk of life-threatening toxicity if a particular drug combination is administered to the patient. One may use conventional means to assess toxicity. Thus, e.g., one may use the toxicity evaluation processes described in U.S. Pat. Nos. 5,736,352 (method and apparatus for determination of the activity of cholesterol oxidase and method and apparatus for evaluation of the toxicity of chemical substances), 6,878,518 (methods for determining steroid responsiveness), and 6,878,522 (methods for the identification of compounds useful for the treatment of disease states mediated by prostaglandin D2). The entire disclosure of each of these United States patents is hereby incorporated by reference into this specification.

In another embodiment, not shown, the lymphocytes, granulocytes, dendritic cells, and CTLs collected from the peripheral blood of a patient in step 26 are used to compare the molecular character of normal cells to that of the diseased cells of the particular patient.

In another embodiment, not shown, the lymphocytes, dendritic cells, and the like, collected from the peripheral blood of a patient in step 26 are used to allow assessment of the capacity of that individual patient to mount an immune response to a given antigen. In one preferred embodiment, the assessment will be used to indicate the likelihood of an immunotherapeutic response. Additionally, or alternatively, one may collect clinical information (from a clinical laboratory) that also may be submitted to database 28.

In one embodiment, the serum of a patient is collected to be used for further analyses. As is known to those skilled in the art, serum is the fluid obtained from blood after it has been allowed to clot; it is also the plasma without fibrogen.

One may use conventional means for collecting the serum from the biological organism. Thus, e.g., one may use one or more of the processes and/or devices disclosed in U.S. Pat. Nos. 4,775,620 (cytokeratin tumor markers and assays for their detection), 5,120,413 (analysis of samples using capillary electrophoresis), 5,159,063 (isolation and characterization of 120 kDa glycoprotein plasma), 5,259,939 (capillary electrophoresis buffer), 5,630,924 (compositions, methods, and apparatus for ultrafast electroseparation analyses), and the like. The disclosure of each of these United States patents is hereby incorporated by reference into this specification.

In one embodiment, in step 26 the white blood cells of the biological organism are collected and analyzed. One may make such collection and analyses by conventional means. Reference may be had, e.g., to U.S. Pat. No. 4,187,979, the entire disclosure of which is hereby incorporated by reference into this specification.

Referring again to FIG. 1, data obtained in steps 18 and/or 20 and/or 22 and/or 24 and/or 26 are preferably conveyed via lines 19 and/or 21 and/or 23 and/or 25 and/or 27 to database 28.

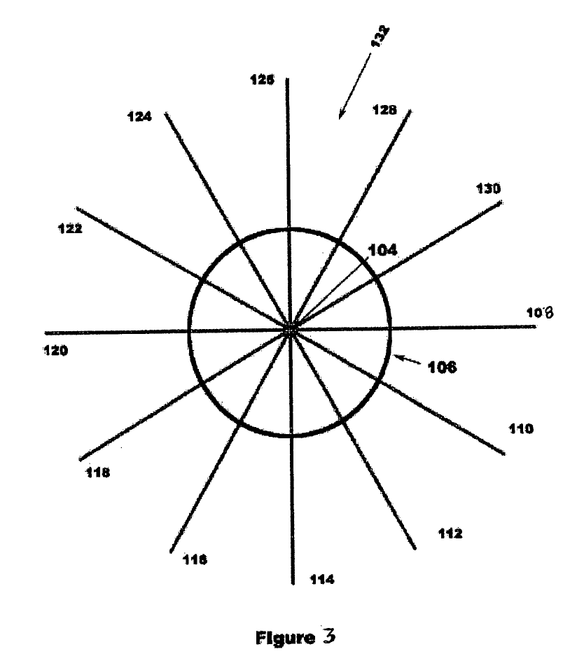

In one embodiment, the database 28 is a relational informatics database in which incoming information is organized according to the “Hankins Medical Mapping System (HaMMS)” and “Hankins coordinates,” as defined below by reference to FIG. 3, which will serve as relational links between samples, diagnoses, treatments, and technologies. The “Hankins Medical Mapping System database” may be constructed in accordance with conventional means disclosed in the prior art. Reference may be had, e.g., to U.S. Pat. Nos. 5,706,498 (gene database retrieval system where a key sequence is compared to database sequences by a dynamic programming device), 5,970,500 (database and system for determining, storing, and displaying gene locus information), 6,023,659 (database system employing protein function hierarchies for viewing biomolecular sequence data), 6,256,647 (method of searching database of three-dimensional protein structures), 6,532,462 (gene expression and evaluation system using a filter table with a gene expression database), and the like. The entire disclosure of each of these United States patents is hereby incorporated by reference into this specification.

By way of illustration and not limitation, U.S. Pat. No. 5,970,500 describes and claims “1. A method of displaying the genetic locus of a biomolecular sequence, the method comprising the following: providing a database including multiple biomolecular sequences, at least some of which represent open reading frames located along a contiguous sequence on an organism's genome; identifying a selected open reading frame; and displaying the selected open reading frame together with adjacent open reading frames located upstream and downstream from said selected open reading frame, wherein the adjacent open reading frames and the selected open reading frame are displayed in the relative positions in which they occur on the contiguous sequence.”

FIG. 3 is a schematic of the first two dimensions of the “Hankins Medical Mapping System” 100 that allows kinetic mapping of life, life's molecules, and life's processes. This system is preferably derived from the data produced in steps 18 and/or 20 and/or 22 and/or 24 and/or 26. The “Hankins Medical Mapping System” 100 fully consists of 5 dimensional representations as is described elsewhere in this specification. . These 5 dimensions allow a complete kinetic mapping of a cell, the cell's molecules, the cell's processes, and the cell's responses to agents in its environment.

Referring again to FIG. 3, the “Hankins Medical Mapping System” 100, in the preferred embodiment depicted, is in the form of a unit circle 106 with its center 104 at the origin of a polar coordinate system. The system depicts the biological cycle of cell differentiation along myriad vectors or radii from a zygote or stem cell 104 to a fully differentiated cell. At the origin of 104 of the medical mapping system is the zygote or stem cell, from which any number of differentiation vectors may radiate. The magnitude of a given vector, which may be less than or equal to 1, describes the extent to which a particular cell lineage has progressed towards full differentiation. The angle of the vector describes the order of the represented cell lineage in the overall progression of the differentiation of the organism: the larger the angle, the later in the progression the given lineage develops.

Referring again to FIG. 3, and as will be apparent to those skilled in the art, the components of coordinate system are not drawn to scale for purposes of ease of illustration.

Referring again to FIG. 3, it will be seen that, in the preferred embodiment depicted, and connected to center 104, are there are twelve radii of differentiation 108, 110, 112, 114, 116, 118, 120, 122, 124, 126, 128, and 130. In one embodiment, these twelve radii of differentiation are separated from each other by about 30 degrees. In the embodiment depicted, radius 126 represents formation of blood islands, the first cellular evidence of the tissue blood. Radii 128 et seq. represent the next organ systems to develop in the fetal development of the organism. As should be readily apparent, the choice of twelve radii of differentiation was made for purposes of illustration; there often are more than twelve radii which will be present in a complete mapping of all of an organism's cell lineages. The radii present will include but not be limited to, e.g., erythrocytes, granulocytes, B cells, T cells, spleen, liver, brain, etc.

Referring to FIG. 3, it will be seen that each of such radii of differentiation 108 et seq. emanate from the origin 104 (at which the zygote/stem cell is located) and radiate toward the periphery 132. The distance between the origin 104 and the periphery 132 represents the space and time over which a cell differentiates from an immature stem cell to a mature cell capable of performing functions for the biological organism. Each of such radii can be divided into units between 0 and 1 that reflect the degree of differentiation.

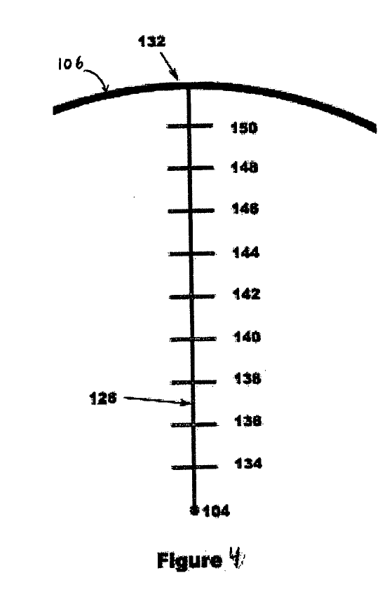

FIG. 4 is a schematic illustration of how one of the radii of differentiation, radius 126, may be divided into, e.g., ten distinct units 134, 136, 138, 140, 142, 144, 146, 148, 150, and 132. In the embodiment depicted in FIG. 4, each of the units 134 et seq. reflects a percentage of the extent to which the development process in question, from the egg/sperm cell has neared completion (at point 132). These units in toto reflect the span of the cell, from origin to death.

Without wishing to be bound to any particular theory, applicant believes that, during the development of a cell, such cell will trace its lineage along a particular line beginning at origin, 104, and developing along a particular line out to the unit circle, 106. As cells develop, they progress as a family or clone of cells in only one direction and remain on a particular line. Knowledge of where a cell is in its particular developmental pathway will provide the necessary information to allow a physician the ability to promote growth of the cell or to terminate the growth of the cell.

In one embodiment, not shown, gene expression can be documented at each of the different radii of differentiation. As is known to those skilled in the art, gene expression is a multistep process, and regulation of the process, by which the product of a gene is synthesized.

As will be apparent to those skilled in the art, the coordinate system depicted in FIG. 3, which is analogous to polar coordinates, can be used to construct a polar map of gene expression, protein expression, drug responsiveness, polymorphisms, single point mutations, additions, and deletions, physiological processes, and the like.

In one embodiment, not shown, a vector rising in the z-coordinate in the base cylindrical medical mapping coordinate system from a point disposed approximately midway in said vector is used to document gene expression. As a cell progresses along its particular developmental pathway (i.e. lineage), different genes will be expressed. When the gene expression, which was observed in Step 20 and/or Step 22 in FIG. 1, is entered into the database 28, the gene expression can be used as a reference point to document a cells' location along its particular developmental pathway.

In the aforementioned embodiment, for the cell which is at such midway point, the genes which are being expressed may be referenced as other first discrete points, and the genes which are not being expressed may be referenced as additional discrete points. Genetic expression, as evidenced e.g. by the functioning receptors on the cell membrane surface and the proteins being generated by the cell and the like, may thus be represented. As a particular cell or group of cells progresses through its development and matures, different genes will be expressed. The genetic expression of the cell is readily observable and may be used as a marker to identify the location of the cell along its developmental pathway. Thus as a cell traverses its specific lineage pathway the degree of gene expression for a particular gene will vary. In one particular embodiment, not shown, the particular cell is an erythrocyte precursor destined to make an erythrocyte and the erythropoietin receptor expression will be at a certain percentage, e.g. 50 percent, of its maximal level of expression relative to certain housekeeping genes, e.g. actin, gap dehydrogenase, and the like, and the globin expression will be at a certain percentage, e.g. 5 percent, of its maximal level of expression relative to the reference housekeeping genes.

In another embodiment, also not shown, a vector rising in a fourth coordinate in the base cylindrical medical mapping coordinate system from at a discrete point and a vector rising from the discrete point are used to document normal or abnormal gene expression. In one embodiment, not shown, for the cell which is at point 140 in FIG. 4 a gene or genes which is/are expressed at a discrete point or points can be shown as being expressed in a normal non-mutated manner. Additionally, an additional gene or genes which is/are expressed at an additional discrete point or points can be shown as being expressed in a mutagenic cancerous manner.

Without wishing to be bound to any theory, applicant believes that sequences deviating from normal may result from somatic point mutations and/or single nucleotide polymorphisms, and/or chromosomal deletions, additions and/or translocations.

In another embodiment, also not shown, a particular cell or group of cells at a particular developmental address is responsive to exposure to various external agents such as chemotherapy drugs, hormones, or other biologicals, radiation or infectious agents, and the like. In one particular embodiment, also not shown, the cell is a chronic myelogenous leukemia (CML); as such the cell is responsive to hemopoietic lineage specific hormones, e.g., erythropoietin, but is not responsive to non-hemopoietic lineage specific hormones, e.g. estrogen.

In another embodiment, also not shown, the cell is an ovarian cancer cell; as such the cell is responsive to ovarian lineage specific hormones, e.g. estrogen, follicle stimulating hormone, and the like, but it is not responsive to non-ovarian lineage specific hormones, e.g. thyrotropin, erythropoietin, and the like.

FIG. 5 illustrates a process 14 (see FIG. 1) for preserving, expanding and further analyzing the physiology or pathophysiology of the tissue sample obtained in step 12 (see FIG. 1) in live or viable state. The process of step 14 of FIG. 1 may comprise the steps of freezing cells (in step 200), and/or constructing a molecular bank of the cells (in step 202), and/or vitrification of the cells (in step 204), and/or constructing primary cell lines (in step 206), and/or using a “scid mouse” (in step 208).

In step 200, the tissue sample may be preserved by freezing it and its cells. This process may be effected by conventional means. Reference may be had to, e.g., U.S. Pat. Nos. 5,102,783 (composition and method for culturing and freezing cells and tissues), 5,958,670 (method of freezing cells and cell-like materials), 6,140,123 (method for conditioning and cryopreserving cells), and the like. The entire disclosure of each of these United States patents is hereby incorporated by reference into this specification.

In step 202, a molecular bank of the cells may be constructed by conventional means. Reference may be had, e.g., to U.S. Pat. Nos. 4,849,349 (genes for biologically active proteins), 5,308,770 (cloning and overexpression of glucose-6-phosphate dehydrogenase from Leuconostoc dextranicanus), 5,656,467 (methods and materials for producing gene libraries), 5,869,295 (methods and materials for producing gene libraries), 6,310,191 (generation of diversity in combinatorial libraries), and the like. The entire disclosure of each of these United States patents is hereby incorporated by reference into this specification.

In one embodiment, in step 202, a gene library is prepared. As is known to those skilled in the art, a gene library is a clone library that contains a large number of representative nucleotide sequences from all sections of the DNA of a given genome; it is a random collection of DNA fragments from a single organism, linked to vectors, and cloned in a suitable host. The DNA from the organism of interest is fragmented (enzymatically or mechanically), the fragments are linked to suitable vectors (plasmids or viruses), the modified vectors are introduced into host cells, and the latter are cloned. A gene library contains both transcribed DNA fragments (exons) as well as nontranscribed fragments (introns, spacer DNA). Retrieval of specific DNA sequences from a gene library frequently involves screening by means of a probe. Reference may be had, e.g., to U.S. Pat. Nos. 4,874,845 (T lymphocyte receptor subunit), 4,966,846 (molecular cloning and expression of a vibrio proteolyticus neutral protease gene), 5,252,475 (methods and vectors for selectively cloning exons), 5,721,110 (methods and compositions useful in the diagnosis and treatment of autoimmune diseases), 6,054,267 (method for screening for enzyme activity), 6,291,161 (method for tapping the immunological repertoire), 6,472,146 (methods for identification on internalizing ligands and identification of known and putative ligands), 6,613,528 (cellulose films for screening), 6,555,315 (screening for novel bioactivities), and the like. The entire disclosure of each of these United States patents is hereby incorporated by reference into this specification.

In one embodiment, and referring again to step 202, a cDNA library is prepared. As is known to those skilled in the art, a cDNA library is a clone library that differs from a gene library in that it contains only transcribed DNA sequences (exons) and no nontranscribed DNA sequences (introns, spacer DNA). It is established by making complementary DNA from a population of cytoplasmic mRNA molecules, using the enzyme RNA-dependent DNA polymerase (reverse transcriptase), converting the single-stranded cDNA to double-stranded DNA, and cloning the latter as in the establishment of a gene library. Reference may be had, e.g., to U.S. Pat. Nos. 5,700,644 (identification of differentially expressed genes), 6,143,528 (method for forming full-length cDNA libraries), 6,174,669 (method for making full-length cDNA libraries), 6,221,585 (method for identifying genes underlying defined phenotypes), 6,607,899 (amplification-based cloning method), and the like. The entire disclosure of each of these United States patents is hereby incorporated by reference into this specification.

Referring again to FIG. 5, and in step 204 thereof, one may preserve the tissue sample and its cells by the process of vitrification. As is know to those skilled in the art, vitrification is an experimental procedure for preserving human organs in which chemicals are added prior to cooling to prevent crystallization of water within and outside the cells, so that, with cooling, the molecules essentially become fixed in place. Reference may be had, e.g., to U.S. Pat. Nos. 4,559,298 (cyroperservation of biological materials in a non-frozen or vitreous state), 5,200,399 (method of protecting biological material from destructive reactions in the dry state), 5,290,765 (method for protecting biological materials form destructive reactions in the dry state), 5,518,878 (cryopreservation of cultured skin or cornea equivalents with agitation), 5,962,214 (method of preparing tissues and cells for vitrification), 6,500,608 (method for vitrification of biological cells), 6,519,954 (cryogenic preservation of biologically active material using high temperature freezing), and the like. The entire disclosure of each of these United States patents is hereby incorporated by reference into this specification.

Referring again to FIG. 5, in step 206 thereof, primary cell lines are prepared by conventional means. As is known to those skilled in the art, a primary culture is a culture that is started from cells, tissues, or organs that are derived directly from an organism, or tissue freshly explanted from the organism. Reference may be had, e.g., to U.S. Pat. Nos. 5,399,493 (methods and compositions for the optimization of human hematopoietic progenitor cell cultures), 5,437,994 (method for the ex vivo replication of stem cells, for the optimization of hematopoietic progenitor cell cultures, and for increasing the metabolism, GM-csf secretion, and/or IL-6 secretion of human stromal cells), 5,474,770 (biological support for cell cultures constituted by plasma proteins coagulated by thrombin, its use in the preparation of keratocyte cultures, their recovery and their transport for therapeutic purposes), 5,602,028 (system for growing multi-layered cell cultures), 5,658,797 (device for the treatment of cell cultures), 5,728,541 (method for preparing cell cultures from biological specimens for chemotherapeutic and other assays), 5,888,816 (cell cultures of and cell culturing method for nontransformed pancreatic, thyroid, and parathyroid cells), and the like. The entire disclosure of each of these United States patents is hereby incorporated by reference into this specification.

In one embodiment of the invention, it is preferred to culture tumor cells by defining the growth requirements (horrnones, growth factors) that select for and propagate the tumor cells and not contaminating fibroblast and other non-tumor stromal cells.

Referring again to FIG. 5, and to step 208 thereof, cells from the tissue sample are implanted into an immunodeficient mouse. One may use any of the immunodeficient mice known to the prior art in this step 208 (Severe Combined Immunodeficient, SCID, or Non Obese Diabetic-SCID, NOD-SCID mice). Reference may be had, e.g., to U.S. Pat. Nos. 5,602,305 (immunodeficient animal model for studying T cell-mediated . . . ), 5,625,127 (extended humanhematopoiesis in a heterologous host), 5,633,426 (in vivo use of human bone marrow for investigation and production), 5,639,939 (chimeric immunocompromised mammal comprising vascularized fetal organ tissue), 5,643,551 (small animal metastasis model), 5.849,998 (transgenic animals expressing a multidrug resistance cDNA), 5,859,307 (mutant RAG-1 deficient animals having no mature B and T lymphocytes), 5,925,802 (functional reconstitution of SCID-bo mice with bovine fetal hematopoietic tissues), 5,986,170 (murine model for human carcinoma), 5,994,617 (engraftment of immune deficient mice with human cells), 6,087,556 (transgenic animals capable of replicating hepatitis viruses and mimicking chronic hepatitis infection in humans), 6,284,239 (murine model for human carcinoma), 6,353,150 (chimeric mammals with human hematopoietic cells), 6,410,824 (animal model for psoriasis for the prevention and treatment of psoriasis in humans), 6,509,514 (chimeric animal model susceptible to human hepatitis C virus infection), 6,620,403 (in vivo chemosensitivity screen for human tumors), and the like. The disclosure of each of these United States patents is hereby incorporated by reference into this specification.

In one embodiment, a “scid mouse” (“severe combined immunodeficient” mouse) is implanted with the cells of the tissue sample. These mice are well known to those in the art and are described, e.g., in U.S. Pat. Nos. 5,994,617 (engraftment of immune-deficient mice with human cells), 6,284,239 (murine model for carcinoma), 6,107,540 (mice models of human prostate cancer progression), 6,639,121 (inducible cancer model to study the molecular basis of host tumor cell interactions in vivo), and the like. The entire disclosure of each of these United States patent applications is hereby incorporated by reference into this specification.

Referring again to FIG. 5, the scid mouse of step 208 may be used for preservation and expansion of the cells (see step 210), and/or tumor modeling (see step 212), and/or serum biomarker analysis (see step 214), and/or the construction of a personalized xenograph (see step 216), and/or hormone requirement analysis (see step 218).

In one embodiment of step 214, purified tumor cells produced in step 16 of FIG. 1 are transplanted in the scid mouse (see step 208 of FIG. 5), the transplanted tumor cells are allowed to grow for a period of up to about one year or more. Serum samples are periodically collected from the implanted mouse, preferably on a monthly basis; and the serum from the transplanted recipient mouse is periodically analyzed by serum proteomics technology. Such serum analysis techniques are well known. By way of illustration, reference may be had, e.g., to U.S. Pat. Nos. 4,115,062 (cancer analysis by serum analysis of glycolipids), 5,223,397 (soluble HLA cross-match), 5,270,169 (detection of HLA antigen-containing immune complexes), 5,482,841 (evaluation of transplant acceptance), 6,019,945 (sample analysis system), and the like. The entire disclosure of each of these United States patents is hereby incorporated by reference into this specification.

Referring again to FIG. 5, and to step 214 thereof, in another embodiment, one may use tumor stem cells that are identified, e.g., by the process described in U.S. Pat. No. 5,994,617 of John E. Dick, the entire disclosure of which is hereby incorporated by reference into this specification.

Referring again to FIG. 1, and in the preferred embodiment depicted therein, information from the database 28 may be used to deduce the developmental address of the single cells (in step 30), and/or to deduce the best therapy for treating a disease condition and/or to discover new therapies (in step 32), and/or to deduce a biomarker panel and/or to thus discover new biomarkers (in step 34). The deduction of the developmental address (in step 30) may lead to lineage specific drug discovery (in step 36), and/or a lineage specific response/diagnostic (in step 38), and/or to a lineage specific screening platform (in step 40).

Referring again to FIG. 1, in step 30, the information from the database 28 is used to deduce the developmental address of normal and/or abnormal cells. In one preferred embodiment, the coordinate system depicted in FIG. 3 is used to deduce the developmental address of normal or abnormal cells.

Referring again to FIG. 1, the developmental address of normal and/or abnormal cells can be used to lineage specific drug discoveries (step 36), and/or lineage specific responses and/or diagnostics (step 38), and/or lineage a specific screening platform (step 40).

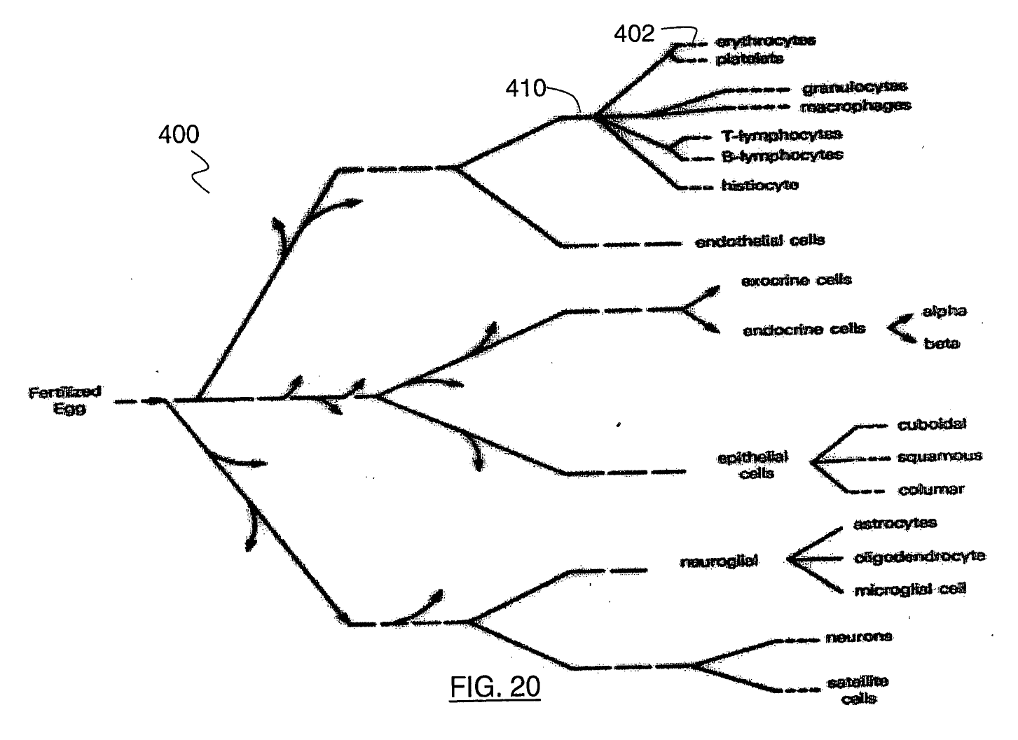

FIG. 20 is a schematic representation of a preferred process 400 for deducing the developmental address of cells that are abnormal. In the preferred embodiment, not shown, the developmental address of abnormal cells is deduced. Abnormal cells maintain four properties of normal cells, viz., hormone sensitivity, the need for specific viability factors to survive, the ability to mature through their lineage pathway, and the exhibition of heterogeneity by the clones of the cells. The observation of these properties is preferred to deduce the developmental address of abnormal cells. The abnormal cells possess a hormone dependence and therefore also require specific viability factors to survive. By way of illustration, if the abnormal cell depicted at reference 402 in FIG. 20 is an erythroleukemia, it will require erythropoietin to survive. If it is a T-cell lymphoid leukemia, it will require interleukin 2 to survive.

By way of further illustration, if the abnormal cell, is a melanoma, it will require a melanocyte lineage specific hormone to survive.

By way of further illustration, if the abnormal cell, not shown, is an ovarian cancer, it will require follicle stimulating hormone to survive.

By way of further illustration, if the abnormal cell, not shown, is FIG. 20 is a thyroid cancer, it will require a thyroid lineage specific factor to survive.

Referring again to FIG. 20 (see element 410), abnormal cells are not blocked from progressing through their specific cell lineage pathway. In one preferred embodiment, not shown, the abnormal cells in question are chronic myeloid leukemia cells exhibiting the Philadelphia chromosome. A normal hemopoietic stem cell would progress along its lineage pathway to produce mature granulocytes, erythrocytes, and the like. The chronic myeloid leukemia cell, in the presence of the proper nutrients and specific viability factors, e.g. erythropoietin, will develop into mature cells.

Referring again to FIG. 20, clones can exhibit heterogeneity. As known to those skilled in the art, a clone is a group of genetically identical cells all descended from a single common ancestral cell by mitosis in eukaryotes or by binary fission in prokaryotes; clone cells also include populations of recombinant DNA molecules all carrying the same inserted sequence. In one preferred embodiment, not depicted herein, the chronic myeloid leukemia cell exhibiting the Philadelphia chromosome will progress along any of three different lineage pathways. This is analogous to the normal hemopoietic stem cell progressing along its lineage pathway to produce mature granulocytes, erythrocytes, and the like. The chronic myeloid leukemia cell will develop into mature cancerous granulocytes, erythrocytes, and the like. Thus the clones of the original chronic myeloid leukemia cell exhibit different characteristics.

Referring again to FIG. 1, in step 32, the developmental address of abnormal and/or normal cells can be used to deduce the best therapy to treat the abnormal cells. In one embodiment, the developmental address of abnormal cells is used to deduce the best therapy to treat the abnormal cells. In one additional embodiment, the developmental address of abnormal and normal cells is used to deduce the best therapy to treat the abnormal cells.

Referring again to FIG. 1, in step 34, the developmental address of normal and/or abnormal cells are used to deduce biomarker panel.

Without wishing to be bound to any theory, applicant believes that the thyroid stimulating hormone (TSH) is required for the viability of thyroid cancer cells and, thus, agents which interfere with TSH hormone and/or its interaction with its receptor will lead to death of thyroid cancer cells.

Without wishing to be bound to any theory, applicant also believes that the FLT-3 ligand is required for the viability of certain acute leukemia cells and, thus, agents which interfere with FLT-3 ligand and/or with the interaction of such ligand with its receptor will lead to the death of such acute leukemia cells.

Again, without wishing to be bound to any particular theory, applicant also believes that the follicle stimulating hormone (FSH) is required for the viability of ovarian cancer cells and, thus, agents which interference with either FSH and/or with the interaction of such hormone with its receptor will lead to the death of ovarian cancer cells.

Additionally, applicant believes that agents that interfere with the ligands for EGF receptor III or EGF receptor IV will result in the death of particular tumor cells which are found to express the genes for these receptors and which display said receptors as a part of the tumor cells.

As is known to those skilled in the art, various agents can interfere with one or more of the aforementioned moieties. Such agents may include, e.g., soluble receptors that compete with the receptors on the cancer cells for the ligand and, after binding with the ligand, may be flushed from a biological system. Such agents may also include, e.g., antibodies against the ligand and/or the receptor including, e.g., antibodies that carry toxic molecules (such as radioactive moieties or cytotoxic moieties). Such agents may also include, e.g., small molecules that bind to the receptor or its ligand and thus compete with the cancer receptor/ligand binding event; such agents also may include antisense molecules that block the synthetic path leading to the receptor at one or more sites, thus leading to the death of the cancer cell.

Improvement upon the Process of U.S. Pat. No. 6,258,553

In this section of the specification, an improvement upon the process described in U.S. Pat. No. 6,258,553 is presented.

U.S. Pat. No. 6,258,553 has two independent claims, claims 1 and 2. Claim 1 of this patent describes: “1. A method of determining the apoptosis-inducing activity of a substance, which comprises: a) measuring the optical density of a first cell culture at more than one time point, wherein the first cell culture was contacted with the substance; b) measuring the optical density of a second cell culture at more than one time point, wherein the second cell culture was not contacted with the substance; and c) determining a net slope, which is the difference between the optical density change over time of the first cell culture and the optical density change over time of the second cell culture; wherein a positive net slope indicates apoptosis-inducing activity of the substance.” claim 2 of this patent describes: “2. A method of determining resistance of cells to the apoptosis-inducing activity of a substance, comprising: a) measuring the optical density of a first cell culture at more than one time point, wherein the first cell culture was contacted with the substance; b) measuring the optical density of a second cell culture at more than one time point, wherein the second cell culture was contacted with the substance and is apoptotically sensitive to the substance; and c) determining a net slope, which is the difference between the optical density change over time of the first cell culture and the optical density change over time of the second cell culture; wherein a positive net slope indicates resistance of the first cell culture to the apoptosis-inducing activity of the substance.”

At column 7 of U.S. Pat. No. 6,258,533, the term “optical density,” as used in such patent, is defined. It is stated that “The step of measuring optical density of the culture is done by measuring absorbance at about 550 to 650 nanometers. The optical densities of the cultures are preferably read after shaking.”

Thus, as the term “optical density” is used in U.S. Pat. No. 6,258,533, it refers to a measurement of absorbance; and the values described in, e.g., the Figures of such patent appear to be absorbance measurements using a light source with a wavelength of from about 550 to about 650 nanometers.

Applicant has discovered that, when he measures the transmittance rather than the absorbance of the “first cell culture” and the “second cell culture,” a more accurate representation of the actual “net slope” is obtained. Without being bound to any particular theory, applicant believes that the “net slope” indicated by the transmittance values is a more sensitive indication of apoptosis than is the “net slope” indicated by the absorbance values.

As is used in this specification, the term transmittance is the ratio of the radiant power transmitted by an object to the incident radiant power; and it may be measured by conventional means. Reference may be had, e.g., to U.S. Pat. Nos. 4,019,819 (optical property measurement and control system), 4,159,874 (optical property measurement system and method), 4,243,319 (optical property measurement system and method), 4,288,160 (optical property measurement system and method), 4,296,319 (watermark detection), 5,175,199 (high transparency silica-titania glass beads, method for making, and light transmission epoxy resin compositions), 5,223,437 (direct fibrinogen assay), 5,670,375 (sample card transport method for biological sample testing machine), 5,587,795 (self-aligning substrate transmittance meter), 5,888,455 (optical reader and sample card transport stations for biological sample testing machine), 5,923,039 (ultraviolet transmittance analyzing method and instrument), 5,971,537 (lens specifying apparatus), 6,040,913 (method to determine light scattering efficiency of pigments), 6,236,460 (method for determining the light scattering efficiency of pigments), 6,320,661 (method for measuring transmittance of optical members), and the like. The entire disclosure of each of these United States patents is hereby incorporated by reference into this specification.

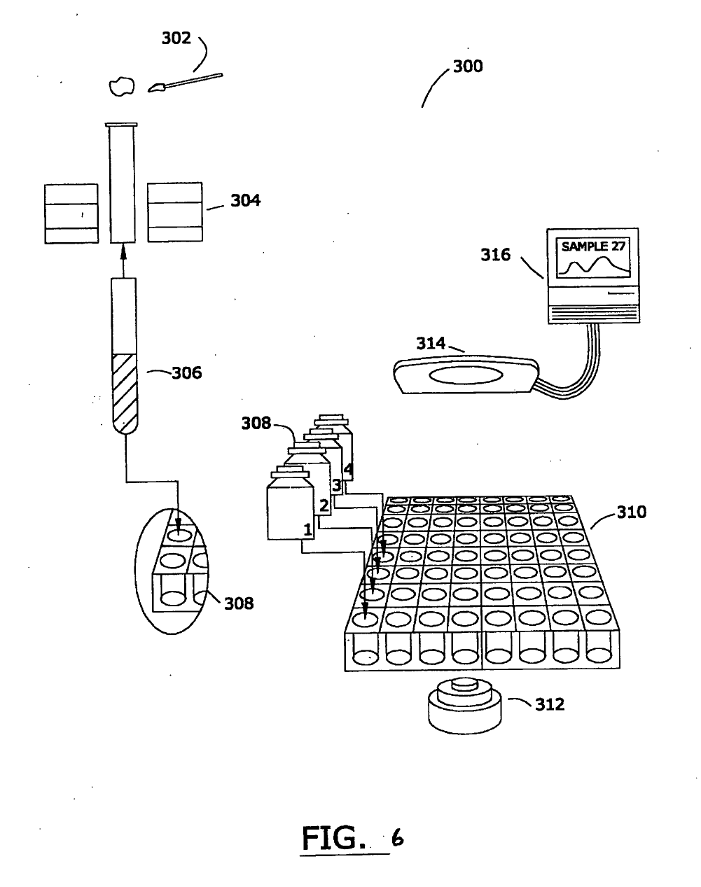

FIG. 6 is a schematic of one preferred process 300. In the embodiment depicted, and in step 302 thereof, a tissue sample is removed by conventional means. One may use, e.g., the cell procurement method described at lines 50 et seq. of Column 14 of U.S. Pat. No. 6,258,553. As is disclosed in such patent, and by way of illustration and not limitation, “Prior to any chemotherapy, a sample of venous blood (e.g., 1-30 ml) or a sample of bone marrow (e.g., 2-20 m: is obtained by direct needle aspiration under sterile conditions. The samples are drawn into a heparinized syringe and diluted with RPMI-1640 medium that contains phenol red. The mononuclear fraction of each sample is isolated by centrifugation using Ficoll-Hypaque. If erythrocytes contaminate the mononuclear cell fraction, then they are removed by treatment with red cell lysis buffer. After washing three times in phosphate buffered saline, an aliquot of the mononuclear cells is analyzed by either light microscopy or flow cytometry for purity and viability. The specific MAb's that recognized the leukemia cells in the diagnostic testing are used to check purity while 7-amino-actinomycin D (7AAD) is used to check viability. If purity and viability are both greater than 90%, then the cells are aliquoted for the present assays and for cryopreservation in RPMI-1640 containing 20% feta. bovine serum and 10% dimethylsulfoxide. Greater than 90% purity and viability would be expected in most cases with a high leukemic cell count in either the blood or bone marrow. If the mononuclear cell fraction purity is less than 90%, then the cells are further purified. T-lymphocytes and monocytes are removed by negative selection using immunomagnetic separation. MAb's to CD2 for T-cell removal and CD14 for monocyte removal and Dynabeads (Dynal, Inc.) are used in those cases in which the diagnostic immunophenotyping shows that the leukemic cells lack these surface antigens. After these immunomagnetic separations, the leukemic cell population will again be tested for purity.”

Referring again to FIG. 6, and in step 304 thereof, a sample is prepared of the cells from the tissue obtained in step 302. This sample may be prepared by conventional means. Thus, e.g., and referring again to U.S. Pat. No. 6,258,553, “Purified leukemic cells are resuspended at from 1.0×105 to 4.0×105 cells/ml in RPMI-1640 medium without phenol red and with 10% fetal calf serum. Note that depending on the microwell plate and the O.D. reader, the concentration of cells may be significantly lower. Aliquots of 250 μliters are cultured in individual wells of a 96-well, flat-bottomed tissue culture microplate.”

In steps 306/308 of the process, the cell samples are placed in specially prepared culture media to which various agents may have been added. By way of illustration, and referring again to U.S. Pat. No. 6,258,553, “Various concentrations of chemotherapeutic agents used to treat acute leukemias are added to duplicate cultures immediately prior to incubation at about 37° C. in 5% CO2 in humidified air. The ranges of concentrations of the agents are based on a) previous reports of apoptosis induced in vitro by these specific agents in either fresh human leukemia cells or human leukemia cell lines and b) pharmacokinetic studies demonstrating that these ranges include concentrations of the parent drugs and/or their active metabolites found in patients following treatment for leukemia. In the present example, the leukemia samples from adults are tested with four agents that are used in their induction and consolidation therapy: 0.1-10.0 μM idarubicin 10,31; 0.01-1.0 μM daunorubicin 11,31; 0.01-10.0 μM cytosine arabinosidel 12,13,32; 0.1-10.0 μg etoposide 11,17,33 and 0.01-1 μM mitoxantrone 16,34,35. For leukemia samples from children, the same concentrations of cytosine arabinoside and etoposide as listed above for adult samples are examined. In place of idarubicin in the adults, daunorubicin at concentrations of 0.01-1 μM are tested. Control wells will receive an equal volume of solvent used for each chemotherapeutic agent. After 30 minutes incubation in humidified air plus 5% CO2, 60 μliters of sterile light mineral oil is layered over each culture, the microplate covered with a lid, and placed in the incubated microplate reader. The O.D. at 600 nm (590-650 nm) of each culture is monitored every five minutes over the ensuing 48 hour period.”

U.S. Pat. No. 6,258,553 discloses that, prior to having their absorbances determined, the cell cultures are agitated. At lines 47-48 of Column 15 such patent, it is disclosed that: “The cultures are shaken with the mixing mode of the incubated microplate reader before each reading is made.” Similarly, at lines 40-44 of Column 7 of U.S. Pat. No. 6,258,553, it is disclosed that “The step of measuring the optical density of the culture is done by measuring absorbance at about 550 to 650 nanometers. The optical densities of the cultures are preferably read after shaking.”

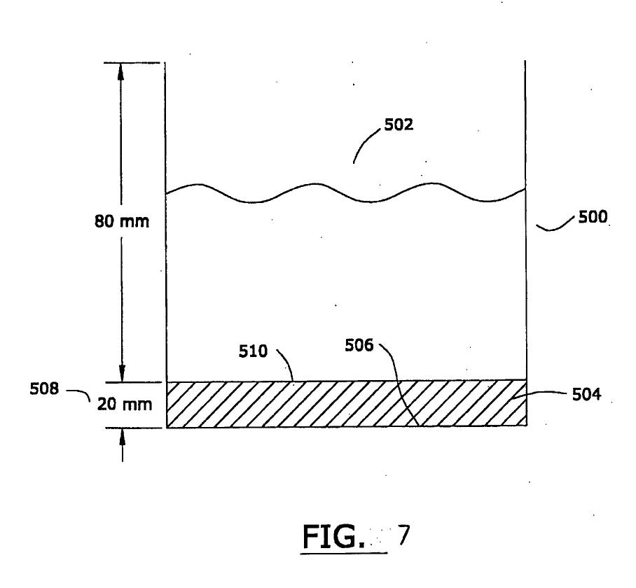

Applicant has discovered that he may provide an improved process by measuring transmittance of cultures that are quiescent rather than agitated. This is illustrated in FIG. 7.

FIG. 7 is a sectional view of a well 500 in which is disposed a culture media 502 that preferably is in a relatively quiescent state. As used herein, the term “relatively quiescent state” means that at least about 90 weight percent of the cellular particulate matter 504 is disposed on the bottom surface 506 of the well and within about the first 20 millimeters distance 508 from such bottom surface 506. Put another way, such wells are typically about 10 centimeters deep, and no more than about 10 weight percent of the cellular particulate matter 504 in the well is disposed above the 20 millimeter line.

Without wishing to be bound to any particular theory, applicant believes that the use of a quiescent state culture medium provides more meaningful data that is more likely to reflect the presence or absence of apoptosis in the cell samples. This is unexpected in view of the clear teaching of U.S. Pat. No. 6,258,553 that a non-quiescent cell culture be used.

Referring again to FIG. 6, and in the preferred embodiment depicted therein, a 96 well microtiter dish 310 is preferably used, and a light source 312 shines light through the samples disposed in such dish. The light source preferably provides light with a wavelength of from 200 about 800 nanometers. In one embodiment, the wavelength provided by the light source is from about 300 to about 700 nanometers. In yet another embodiment, the wavelength provided by the light source is from about 340 to about 660 nanometers.

In the embodiment depicted in FIG. 6, light is being transmitted through a sample 27. The transmitted light is detected by sensor 314, and this information is continually transmitted to controller 316.

In one embodiment, a SpectraMax 340 microplate reader is used for the analyses illustrated in FIG. 6. This microplate reader may be purchased, e.g., from GMI, Inc. of 6551 Jansen Avenue, N.E., Suite 202, Albertville, Minn.

One may use other microplate readers such as, e.g., those disclosed in U.S. Pat. Nos. D404,140 (microplate reader), 4,892,409 (photometric apparatus for multiwell plates having a positionable lens assembly), 5,766,875 (metabolic monitoring of cells in a microplate reader), 5,784,152 (tunable excitation and/or tunable detection microplate reader), and the like. The entire disclosure of each of these United States patents is hereby incorporated by reference into this specification.

Referring again to FIG. 6, and in the preferred embodiment depicted therein, the transmittance values measured by sensor 314 may be converted into optical density values in accordance with the formula: D_{w}=−log₁₀T_{w}, wherein T_{w}, is the measured transmittance of the sample measured at the specified wavelength (w) of the light used, and D_{w} is the optical density of the sample at the specified wavelength of the light used.

As will be apparent to those skilled in the art, although the U.S. Pat. No. 6,258,553 claims to be measuring the optical density of a sample, what in fact it clearly is measuring is only the absorbance of such sample at a specified wavelength. One cannot determine the optical density of a sample merely by measuring the degree to which it absorbs light of a certain wavelength.

Thus, applicant's process allows one to measure the true optical density of a sample rather than mere absorbance. Once such optical density has been measured, one can then plot optical density versus time (as illustrated in the Figures of U.S. Pat. No. 6,258,553) and obtain a true rather than a distorted indication of cell apoptosis.

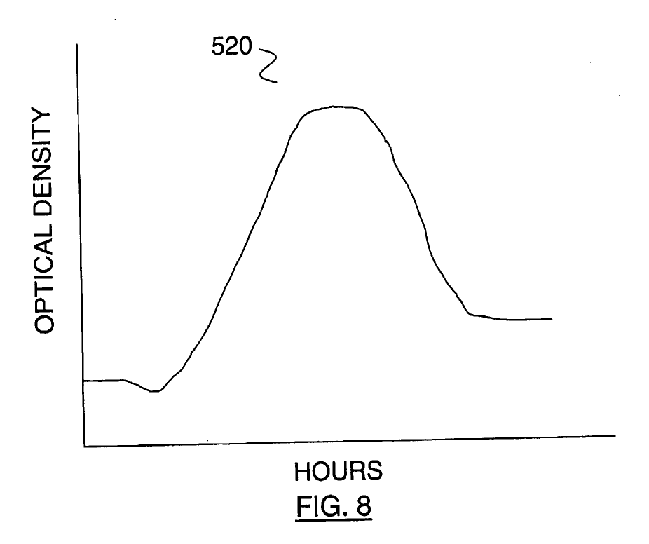

FIGS. 8 through 13 are schematic representation of graphs of data representing the relationships of optical density versus time for various biological systems. In one preferred embodiment, as is shown in FIGS. 8-13 the plots of optical density versus time which are obtained from applicant's process allow one to measure other cell activities besides apoptosis. FIGS. 8 through 13 provide plots of optical density versus time for several cell samples from steps 306/308 from FIG. 6.

By way of illustration, FIG. 8 is a representative graph 520 illustrating cells in the medium undergoing apoptosis, as is evident from the initial increase in optical density during membrane blebbing followed by a decrease in optical density during the breakup of the cells.

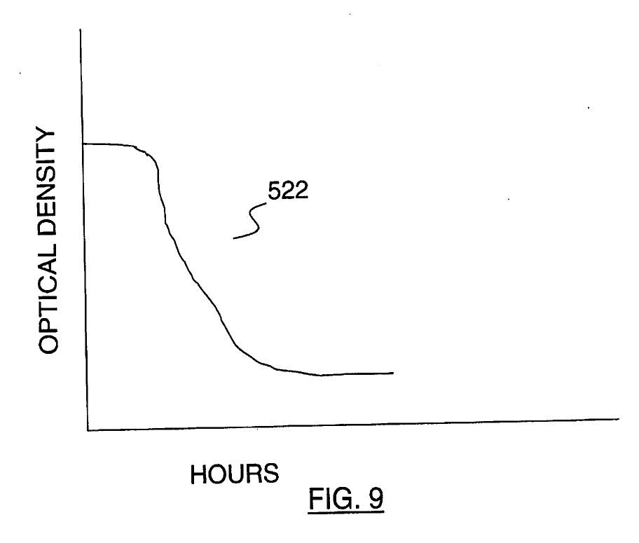

By way of further illustration, FIG. 9 is a representative graph 522 illustrating the behavior of cells in the medium undergoing necrosis, as is evident from the initial decrease in optical density as cells die followed by the optical density remaining constant after there are no more cells to break up.

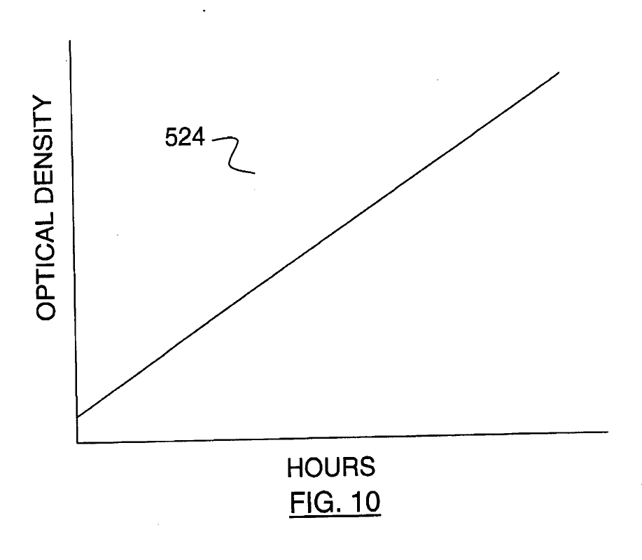

Referring to FIG. 10, this Figure is a representative graph 524 representing cells in the medium undergoing proliferation, as is evident from the continuous increase in optical density which applicant believes is indicative of cell growth and replication within the medium.

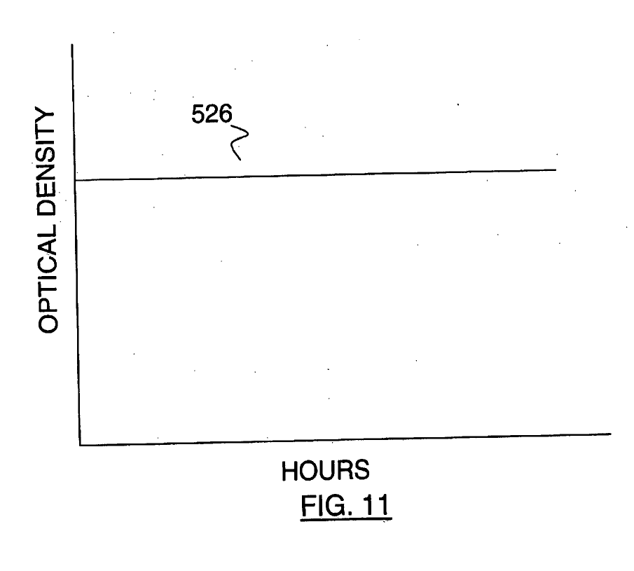

Referring to FIG. 11, graph 526 represents cells in the medium undergoing cytostasis as is evident by the constant nature of the optical density. This is indicative that the cell population within the medium remains constant, i.e., there is neither an appreciable increase nor appreciable decrease in viable cells within the medium.

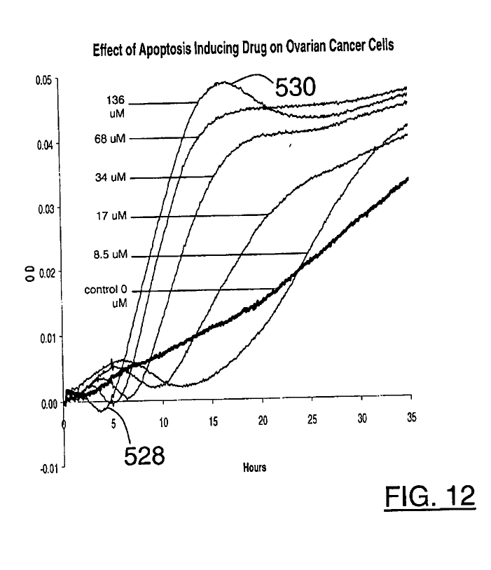

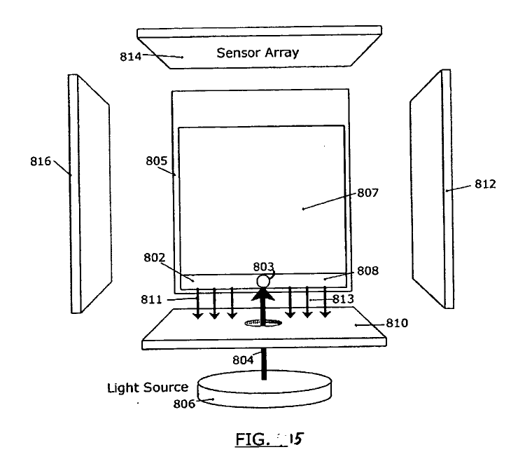

Referring to FIGS. 12, a dip 528 in optical density, 528 is observed prior to the development of the “apoptosis peak”. Without wishing to be bound to any particular theory, applicant believes that the dip in optical density at point 528 is due to a decrease in forward light scatter caused by the shrinking of cells, possibly by water loss from the cells prior to the blebbing process. One may arrange the receptor cell(s) 814 in FIG. 15 to kinetically measure only forward scatter. By measuring only the forward scatter, the drug dose dependent dip in optical density may be measured; and one may develop a novel assay for apoptosis that has superiority over any current assays, e.g. a 2 hour assay for cis platin on Ovarian Cancer cells (see point 528 of the graph of FIG. 12).

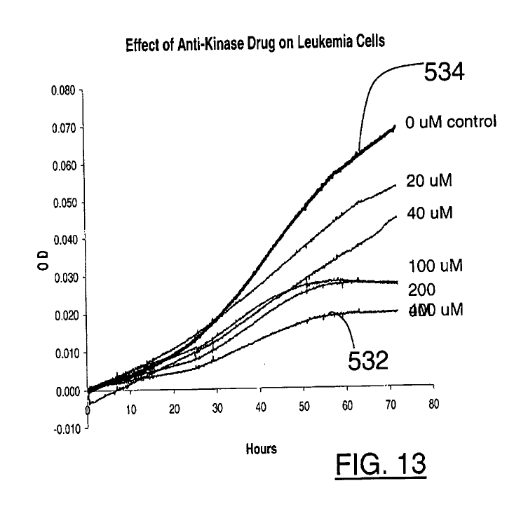

FIG. 13, is a graphical representation of the dose-dependent decrease in measured optical density from the control curve for certain drugs, e.g. imatinib mesylate, which cause apoptosis much more slowly that cytotoxic drugs, e.g. idarubicin. Such drugs do not produce an “apoptosis peak” in the KOR assay. The activity of such drugs can be quantitated by the relative decrease in the experimental slopes, 532 relative to the control slope 534. In one preferred embodiment, shown in FIG. 13, the dose-dependent decrease in slope generated by adding imatinib mesylate, an anti-kinase drug rather than a cytotoxic drug, to K562 tumor cells derived from a patient with chronic myelogenous leukemia.

A Preferred Process Involving Solid Tumors

U.S. Pat. Nos. 6,077,684 and 6,258,553 disclose assays for measuring apoptosis in cell cultures. In the processes described in these patents, cell procurement is conducted by obtaining samples of cells in cell culture media.

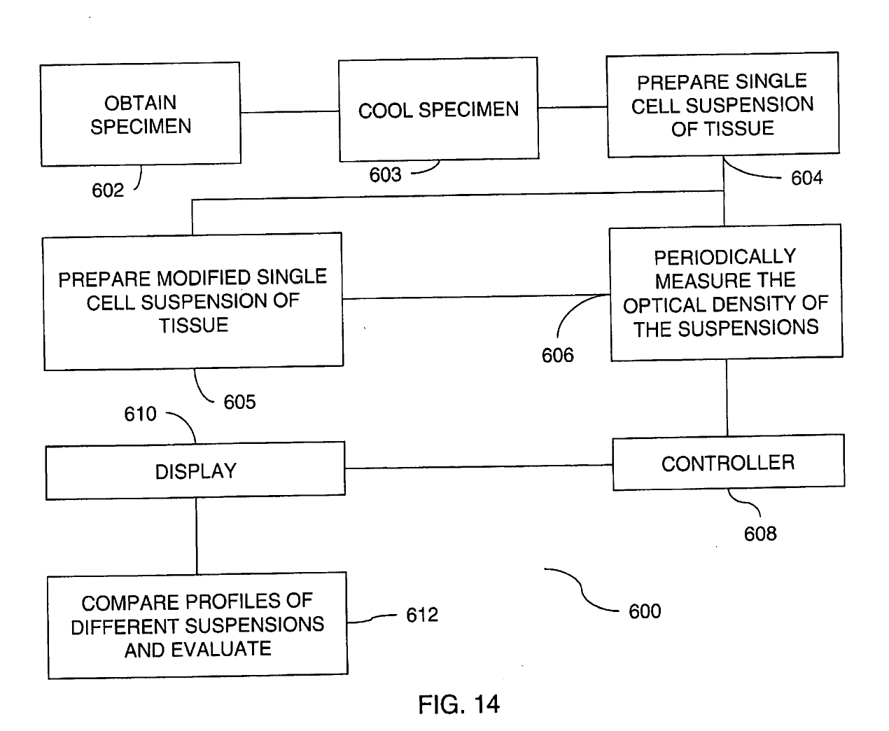

Nowhere in U.S. Pat. Nos. 6,077,684 and 6,258,553 is there any disclosure as to how soon after the cells are procured they are subjected to the specified assay. Applicant has discovered that, in his assay (which utilizes optical density rather than absorbance measurements), it is highly advantageous to use freshly explanted cells in the assay, especially when the cells are derived from solid tumors. This process 600 of conducting this step is illustrated in FIG. 14.

Referring to FIG. 14, and in step 602, a specimen of tissue is obtained in step 602. Such a specimen is often obtained surgically by conventional means. With regard to the remainder of the discussion relating to process 600, it will be assumed that specimen obtained is from a solid tumor; it will be apparent, however, that other sources for the specimen also may be used.

In step 604 of the process, a single cell suspension of the tumor is prepared by conventional means. Thus, e.g., one may use various methods of tissue desegregation such as, e.g., mincing into small pieces. Reference may be had, e.g., to U.S. Pat. Nos. 5,744,363 (method for establishing a tumor-cell line by preparing single-cell suspension of tumor cells from tumor biopsies), 6,114,128 (method and kit for predicting the therapeutic response of a drug against a malignant tumor), 6,448,030 (method for predicting the efficacy of anti-cancer drugs), and the like. The entire disclosure of each of these United States patents is hereby incorporated by reference into this specification.

In one embodiment, the single cell suspension of the tumor cell is preferably prepared within less than about 60 hours of obtaining the tissue sample and, more preferably, within less than about 48 hours of obtaining the sample. In one embodiment, the single cell suspension is prepared from 0.1 to 10 hours after obtaining the tissue sample.

Prior to the time the single cell suspension is prepared, the tissue sample is preferably maintained at a temperature of from about 3 to about 15 degrees Celsius, by cooling (see step 603). It is critical, however, that the tissue sample not be allowed to freeze. In one embodiment, the tissue sample is maintained at from about 4 to about 10 degrees Celsius.

In one embodiment, during each of steps 602, 603, and 604, the source of the specimen and/or the specimen and/or the single cell suspension preferably is exposed to an oxygen-containing gas, such as air.

In another embodiment, during each of steps 602, 603, and 604, the source of the specimen and/or the specimen and/or the single cell suspension is exposed to an oxygen-deficient atmosphere.

In one embodiment, during one or more of the steps 602, 603, and/or 604, the source of the specimen and/or the specimen and/or the single cell suspension is bathed with a solution containing one or more nutrients such as, e.g., glucose, amino acid(s), protein(s), serum, and the like.

In step 606 of the process, the optical density of the cell suspension is periodically measured, as discussed elsewhere in this specification and in U.S. Pat. Nos. 6,258,553 and 6,077,684.

In step 605 of the process, which may be optional, one may prepare other “modified” single cell suspensions that vary from the suspension 604 in that they contain additional agents, or different agents, or different cells, etc. Thus, e.g., different cell suspensions may contain different concentrations of different chemotherapeutic agents and/or different growth factors and/or different concentrations of such agents and/or factors and/or different combinations of such agents and/or factors. By testing a multiplicity of such combinations, the optimal therapy for a particular malignant tissue may be determined. Alternatively, the optimal growth conditions for at tumor may be determined and thus, lead to means for preventing such growth conditions.

The optical densities of these other, “modified cell suspensions” also are preferably periodically measured in, e.g., a microtiter culture dish assembly. This information is preferably continually fed to controller 608, which continually preferably generates optical density profiles of each of the samples. on a display 610. In one embodiment, the instantaneous changes thus displayed provide information on, e.g., the time when one should add growth agents.

These multiple profiles will enable one to determine when one or more agents should be added, whether one or more agents should be added, the sequence of adding one or more agents, the optimal concentrations and combinations of such agents, and the time course of events subsequent to the addition. This may be done in step 612, where a comparison of profiles made of cell suspensions under different conditions and/or of cell suspensions under similar conditions but with different agents, may be made.

Another Preferred Assay Process

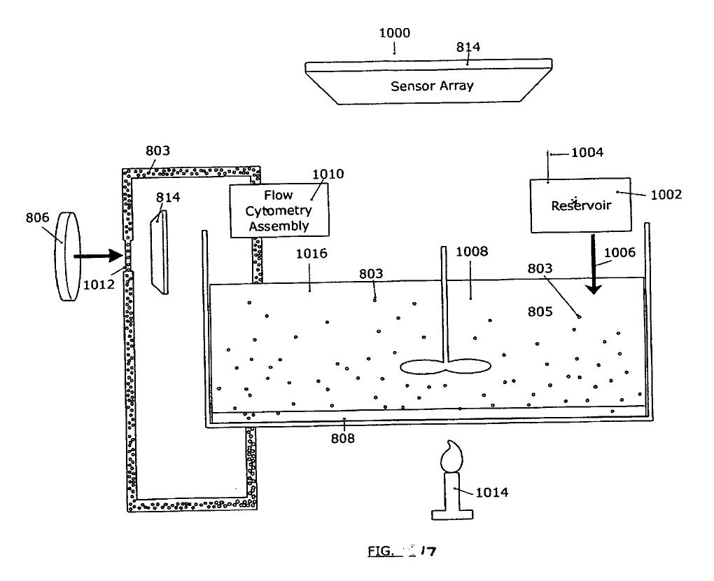

FIG. 15 is a schematic illustration of an assay process 800 that is adapted to determine the kinetic changes in absorbance and/or transmittance and/or optical density and/or light scattering of a particular cell sample. Referring to FIG. 15, and also to FIG. 17, and in the preferred embodiment depicted therein, a medium comprised of the single cells isolated in step 16 of FIG. 1 is preferably fed into a reservoir 1002 by means of line 1004.

In one embodiment, and referring to FIG. 15, a beam of light 804 impacts a cell 803 within a cell medium.

In another embodiment, cell or cells 803 are malignant, and it/they are contacted with light rays 804 emitted by one or more light sources 806 (see FIG. 15). In the embodiment depicted, the cells 803 are disposed in a culture medium 807 which, in turn, is preferably disposed in a culture well 805. As will be apparent, these elements are not drawn to scale to facilitate ease of comprehension.

In the preferred embodiment depicted in FIG. 15, the light rays 804 are preferably emitted substantially perpendicularly to the layer of cells 803. The light source may be one or more of light sources 312 depicted in FIG. 6.

The amount of light emitted by light source 806 is preferably measured by sensor 810, which also determines the amount of light that is transmitted from sensor 810 through to cell(s) 803. Additionally, the sensor 810 measures the amount of light that is reflected back to sensor 810 (see rays 812, 814, and 816).

The sensor 810 may be adapted, e.g., to measure the amount of light scattering. Means for measuring such light scattering are well known to those skilled in the art. Reference may be had, e.g., to U.S. Pat. Nos. 4,915,501 (device for measuring the light scattering of biological cells), 4,923,298 (device for measuring the speed of moving light scattering objects), 4,979,818 (apparatus for measuring movement of light scattering bodies in an object), 5,057,695 (method of measuring the inside information of substance with the use of light scattering), 5,113,083 (light scattering measuring apparatus using a photodetector mounted on a rotary stand), 5,239,185 (method and equipment for measuring absorbance of light scattering materials), 5,481,113 (method for measuring concentrations of components with light scattering), 5,712,167 (method of measuring Amadori compound by light scattering), 5,844,239 (optical measuring apparatus for lights scattering), 5,870,188 (measuring method by light scattering), 6,697,652 (fluorescence, reflectance, and light scattering spectroscopy for measuring tissue), 6,750,967 (light scattering measuring probe), 6,833,918 (light scattering particle size distribution measuring apparatus), and the like. The entire disclosure of each of these United States patents is hereby incorporated by reference into this specification.

In one preferred embodiment, one may use the measuring devices disclosed in U.S. Pat. Nos. 4,673,288 (flow cytometry) and 4,818,103 (flow cytometry), the entire disclosure of which is hereby incorporated by reference into this specification.

U.S. Pat. No. 4,673,288 discloses and claims (see claim 1) “1. A flow transducer comprising means defining an aperture having an axis, said aperture having at least one flat side, means defining an inlet chamber and an outlet chamber immediately adjacent the aperture along its axis, said inlet and outlet chambers having walls disposed at an angle of at least 5° relative to the plane of the aperture, said inlet and outlet chambers at a distance from the aperture of twice the width of the aperture in a plane through its axis having cross-sectional areas at least 10 times the cross-sectional area of said aperture.” U.S. Pat. No. 4,818,103 discloses and claims (also see claim 1) “1. A flow transducer comprising means defining an aperture having an axis, said aperture having at least one flat side, means defining an inlet chamber and an outlet chamber immediately adjacent the aperture along its axis, at least one of said inlet and outlet chamber having walls disposed at an angle of at least 5° relative to the plane of the aperture, said at least one of said inlet and outlet chambers at a distance from the aperture of twice the width of the aperture in a plane through its axis having a cross-section area at least 10 times the cross-sectional area of said aperture.”

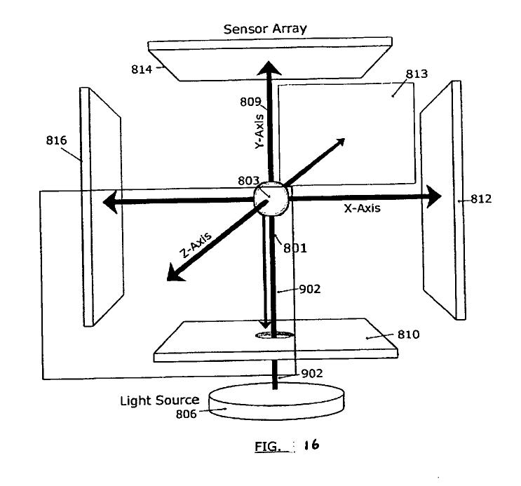

In one embodiment, the devices of U.S. Pat. Nos. 4,673,288 and/or 4,818,103 are adapted to make the kinetic measurements described in FIGS. 15 and 16.

Referring again to FIG. 15, and in the preferred embodiment depicted therein, it will be seen that, in addition to sensor array 810, there are also preferably present sensor arrays 812, 814, 816, and others. These sensor arrays are preferably comprised of sensor means for measuring light scattering, optical density, absorbance, transmittance, and other energy-related properties such as, e.g., temperature, pressure, etc. As will be apparent, in the kinetic process depicted in FIG. 15, a series of graphs can be constructed showing the effect of any particular agent upon any one or more of the physical properties of the cell layer and/or its chemical properties and/or its optical properties and/or its biological properties and/or its biochemical and/or any other of its properties.

In one preferred embodiment, hinted at in FIG. 15, a multiplicity of sensors 810/812/814/816 et seq. are disposed circumferentially around the culture chamber 805 in a 360 degree orientation vis-à-vis such chamber 805 such that light emitted from such chamber in any direction or any axis can be captured by one or more of such sensors. This concept is illustrated schematically in FIG. 16.

FIG. 16 illustrates what happens when a quantum of light 801 contacts a cell 803. Some of the light 811 is reflected back directly to the sensor array 810 (see FIG. 5, and also see FIG. 16) The light 811 that is reflected back to the sensor 810 is referred to as back light scatter in this specification. The change in back light scatter over time may be measured by the process of this invention.

Referring again to FIG. 16, a portion of the quantum of light that impinges upon cell 803 is absorbed by such cell 803. By measuring and monitoring the total amount of light that impacts cell 803 and deducting the amount of light that is either transmitted and/or scattered, one can continually determine the amount of light 801 that is absorbed. The change in absorbance over time may be measured by the process of this invention.

Referring again to FIG. 16, a portion 809 of the quantum of light that impinges upon cell 803 is transmitted through said cell is a direction that is substantially parallel to the incoming quantum of light 801. One thus can continually monitor the amount of light that is transmitted by cell 803, and the change in transmittance over time may be measured by the process of this invention.

Referring again to FIG. 16, a certain amount of the light 801 that impacts cell 803 will be side scattered in the “x-axis) substantially perpendicularly to the direction of the incoming light 801. The change in side scattering over time may be measured by the process of this invention.

Similarly, one may measure the amount of light scattered in the z axis, which light will be perpendicular to the light in the x-axis and/or the y-axis. Many other different parameters also may be measured by specifying, e.g., a particular point in the x,y,z coordinate system and determining how the light at that point varies in time.

The processes depicted in FIGS. 15 and 16 measure a sample of cells that are viable and, thus, may be changing their properties. In the embodiment depicted in FIG. 17, a process is provided for measuring these same cells when other parameters are varied, such as, e.g., their concentrations.

Referring again to FIG. 15, and in the preferred embodiment depicted therein, the cell chamber 805 is preferably comprised of an agent, such as a chemotherapeutic agent, a hormone, an infectious agent, etc., that may affect the viability of the cell 803. However, the system depicted in FIG. 15 is somewhat static in that the concentration of such agent, and/or the concentration of the cell 803, often does not vary very much.

In life, however, the situation is often much more dynamic. An agent that is added to a biological system changes its concentration as it contacts bodily tissue, and the bodily tissue, especially if it is mobile, also often changes its concentration. Thus, the process depicted in FIG. 17 allows one to monitor the kinetic changes in a system over time as one or more of such concentration and/or other properties are varied.

FIG. 17 illustrates a continuous assay system 1000 that is adapted to determine the changes in a dynamic system, in the embodiment depicted, one or more cell viability agents (such as, e.g., cytotoxic agents like paclitaxel) may be added to reservoir 1002 via line 1004, and one or more of the material in reservoir 804 may be added to chamber 805 (see FIG. 15) via line 1006. In a living biological system, the concentration of, e.g., cytotoxic agents is not necessarily static, and the device of FIG. 17 allows you to test the effects of changes in such agents.

Similarly, in a living system, the cells 803 are not necessarily quiescent. The use of a mixer 1008 allows one to stir such cells 803.

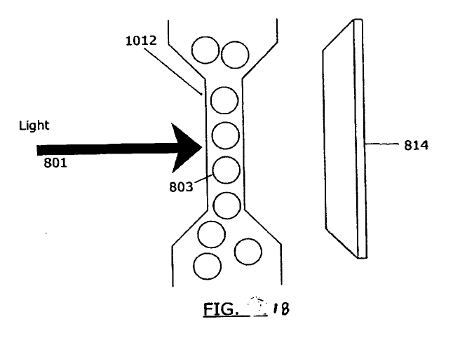

The use of a flow cytometer assembly 1010 allows one to continually move a portion of the cells in the chamber 805 past a single cell inspection station described in greater detail by reference to FIG. 18.

The use of a Bunsen burner, 1014, allows one to change the temperature conditions the cell 803 is subjected to. Similarly, gas can be bubbled into the system via line 1016 to vary the oxygen content of the system.

In the preferred embodiment depicted in FIG. 18, the cells 803 are preferably contacted with light quanta 801, and the responses of such cells 803, in the x and /or y and/or y directions, or at any point in the x, y, z coordinate system, is then determined. In one aspect of this embodiment, it is preferred to contact light 801 with a collection of single cells 803, at point 1012.

Another Preferred Embodiment

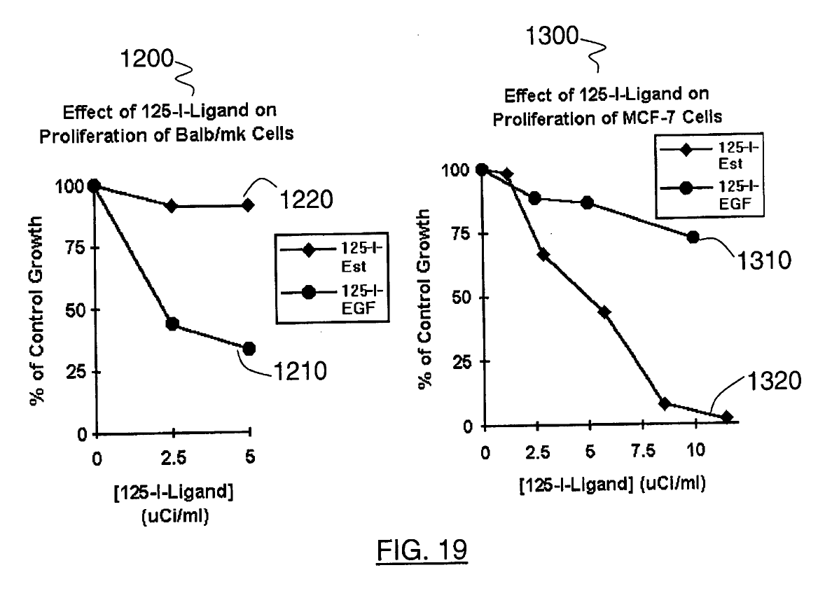

FIG. 19 is a representation of the results of exposing cells to various lineage specific hormones. In one particular embodiment, see chart 1200, epithelial carcinoma cells, 1210, and ovarian cancer cells, 1220, were exposed to radiolabeled epithelial growth factor (EGF). As known to those skilled in the art, a radiolabeled ligand can be used to kill cells that possess receptors for the particular ligand. Reference may be had to U.S. Pat. Nos. 6,565,827 (radioimmunotherapy of lymphoma using anti-CD20 antibodies), 6,287,537 (radioimmunotherapy of lymphoma using anti-CD20 antibodies), 6,015,542 (radioimmunotherapy of lymphoma using anti-CD20 antibodies), and 5,843,398 (radioimmunotherapy of lymphoma using anti-CD 20 antibodies). The entire disclosure of these United States patents are hereby incorporated by reference into this specification. As is readily apparent, more than 50 percent of the epithelial carcinoma cells are killed upon exposure to the radiolabeled epithelial growth factor but less than 10 percent of the ovarian cancer cells are killed upon exposure.

In another embodiment (see graph 1300) epithelial cells, 1310, and ovarian cancer cells, 1320, are exposed to radiolabeled estrogen. Over 90 percent of the ovarian cancer cells are killed upon exposure to the radiolabeled estrogen but less than 30 percent of the epithelial carcinoma cells are killed upon exposure.

Another Preferred Embodiment

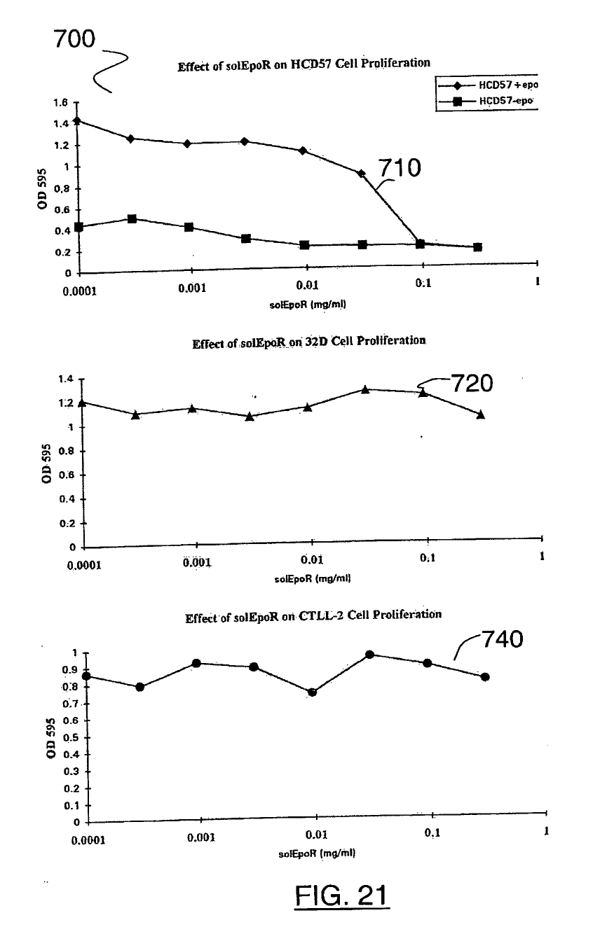

FIG. 21 is a representation of the results of exposing cells to various lineage specific hormone inhibitors 700. In one particular embodiment, depicted in graph 710, a soluble receptor, which will bind free hormone, e.g. erythropoietin, is added to cell cultures of erythroleukemia cells and brings about the death of these cells by depriving them of their essential viability hormone. In this embodiment, adding the soluble receptor which binds erythropoietin does not induce the death of cells of other specific cell lineages, e.g. myeloid cancer cells, 720, and lymphoid cancer cells, 740, and the like.

1. A process for identifying the developmental history of a cell, comprising the steps of:
(a) obtaining a tissue sample from a living biological organism,
(c) disaggregating said tissue sample to produce disaggregated fragments of tissue sample whose maximum dimension is less than about 5 millimeters, wherein said tissue sample is disaggregated within about 10 minutes of the time said tissue sample is obtained from said biological organism, and
(c) disposing said disaggregated tissue fragments in a sterile environment within a container, wherein said sterile environment is comprised of oxygen and a solution comprised of at least one cell type specific viability factor.

2. The process as recited in claim 1, wherein said biological organism is an animal.

3. The process as recited in claim 2, wherein said animal is a human being.

4. The process as recited in claim 1, wherein said tissue sample is obtained from a malignant tumor.

5. The process as recited in claim 4, wherein said viability factor is a viability hormone.

6. The process as recited in claim 5, wherein said viability hormone is a stem cell viability factor.

7. The process as recited in claim 5, wherein said viability hormone is erythropoietin.

8. The process as recited in claim 5, wherein said viability hormone is follicle stimulating hormone.

9. The process as recited in claim 5, wherein said viability hormone is melanocyte stimulating hormone.

10. The process as recited in claim 5, wherein said viability hormone is thyrothropin.

11. The process as recited in claim 5, wherein said viability hormone is epidermal growth factor.

12. The process as recited in claim 5, wherein said viability hormone is present in a viability medium at a concentration of from about 0.01 to about 10 micrograms per milliliter.

13. The process as recited in claim 1, wherein said tissue sample is at least about 90 percent diagnostically pure.

14. The process as recited in claim 1, wherein said tissue sample is disaggregated into fragments of tissue sample whose maximum dimension is less than about 2 millimeters.

15. The process as recited in claim 14, wherein a single cell suspension is prepared from at least one disaggregated fragment of tissue sample.

16. The process as recited in claim 1, wherein said sterile environment is comprised of an oxygen-containing gas comprising at least about 95 volume percent of oxygen.

17. The process as recited in claim 15, wherein the phenotype of said single cell in said single cell suspension is characterized.

18. The process as recited in claim 17, wherein said phenotype is the cellular phenotype of said single cell.

19. The process as recited in claim 17, wherein said phenotype is the molecular phenotype of said single cell.

20. The process as recited in claim 17, wherein said phenotype is the lineage phenotype of said single cell.
